(19) United States
(12) Patent Application Publication
(10) Pub. No.: US 2026/0217674 A1
(43) Pub. Date: Jul. 30, 2026

(54) NEW ACETYLCHOLINESTERASE INHIBITORS AND USES THEREOF FOR PREVENTING AND TREATING COMPULSIVE DISORDERS AND NEURODEGENERATIVE DISORDERS

(71) Applicants: UNIVERSITE DE MCGILL, Montreal, Quebec (CA); SORBONNE UNIVERSITE, Paris (FR); CENTRE NATIONAL DE LA RECHERCHE SCIENTIFIQUE (CNRS), Paris (FR)

(72) Inventors: Salah EL MESTIKAWY, Montreal QC (CA); Nicolas Pietrancosta, Paris (FR); Mathieu FAVIER, Vailhauques (FR)

(73) Assignees: UNIVERSITE DE MCGILL, Montreal, Quebec (CA); SORBONNE UNIVERSITE, Paris (FR); CENTRE NATIONAL DE LA RECHERCHE SCIENTIFIQUE (CNRS), Paris (FR)

(21) Appl. No.: 19/149,239

(22) PCT Filed: Feb. 2, 2024
(86) PCT No.: PCT/EP2024/052658

§ 371 Date: Jul. 18, 2025

(30) Foreign Application Priority Data
Feb. 2, 2023 (WO) PCT/IB2023/000052

(51) Int. Cl. C07D 401/12; A61K 31/4709; A61K 31/4725; A61P 25/00

(52) U.S. Cl. CPC C07D 401/12; A61K 31/4709; A61K 31/4725; A61P 25/00

(57) ABSTRACT

The present invention relates to a compound of formula (I), or a pharmaceutically acceptable salt and/or solvate thereof. The present invention also relates to the use of the compound of formula (I) as a drug, in particular for preventing or treating compulsive disorders and related behaviors, including addiction, obsessive-compulsive disorders and eating disorders, and neurodegenerative disorders such as Parkinson's disease or Alzheimer's disease.

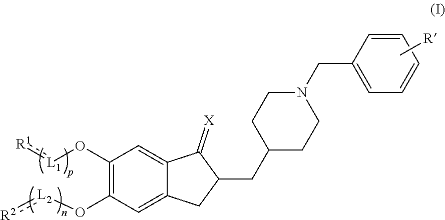

### FILED OF THE INVENTION

The present invention relates to novel acetylcholinesterase inhibitors, particularly useful in the prevention and/or the treatment of acetylcholine-related disorders, such as compulsive disorders, including eating disorders, addiction, obsessive compulsive disorders and neurogenerative disorders.

### BACKGROUND OF THE INVENTION

Acetylcholine (ACh) is a major neuromodulator of the striatal network and the key transmitter at the neuromuscular junction. After its exocytotic release, the action of ACh is rapidly stopped by its hydrolysis that is catalyzed by an enzyme named acetylcholinesterase (AChE). AChE inhibitors (AChEI) inhibit this reaction and prevent the breaking down of ACh into choline and acetate. Thereby, AChEI increases the extracellular levels and duration of action of ACh both in the central nervous system and at the neuromuscular junction.

The Applicant has recently discovered that striatal ACh also play a major role in compulsive disorders, in particular in eating disorders (Favier, et al., The Journal of Clinical Investigation, 2020, 130, 12, p. 6616-6630). Eating disorders such as anorexia nervosa and bulimia nervosa, in fully syndromic and subthreshold forms affect up to 10% of the population. Anorexia nervosa has the highest mortality rates of all mental-health diseases (=5% per decade). Eating disorders result from aberrant processing of habitual behavior. The understanding of the neuronal basis of anorexia nervosa is currently very limited and consequently there are no specific biological nor pharmacological treatments for this dramatic condition.

Cholinergic interneurons are pivotal regulators of the striatum and of habits formation. To investigate the role of ACh in habitual behaviors, the Applicant specifically silenced ACh signaling in the striatum in a mouse model, with a deletion of the vesicular acetylcholine transporter (VAChTcKO mice). The applicant then established that VAChTcKO mice decreased ACh transmission and made mice more prone to excessive habits. To understand if these excessive habits could lead to dysfunctional eating, the Applicant then used a model of pathological eating in VAChTcKO mice: the activity-based anorexia model (ABA, a model of anorexia nervosa (Klenotich and Dulawa, 2012)). In the ABA model, the mutant mice lacking ACh in the striatum were more prone to self-starvation than control mice.

Based on these unexpected findings, the inventors hypothesized that acetylcholinesterase inhibitors could be useful in preventing and/or treating compulsive disorders.

Although several acetylcholinesterase inhibitors are currently available on the market, especially targeting Alzheimer's disease, they often cause severe side effects (dizziness, nausea and vomiting, heart issues, etc.).

Therefore, there is thus a constant need for novel acetylcholinesterase inhibitors with improved affinity for AChE and limited peripherical effect.

### SUMMARY OF THE PRESENT INVENTION

In this context, the inventors have surprisingly designed and developed new acetylcholinesterase inhibitors (AChEI) that are exclusively active in the brain (and not in the body). This mode of action therefore limits neuromuscular action of AChEI and unwanted peripheral side effects. These new compounds were shown to be useful in the prevention and/or treatment of compulsive disorders, especially eating disorders such as anorexia.

According to a first aspect, the present invention relates to a compound of formula (I)

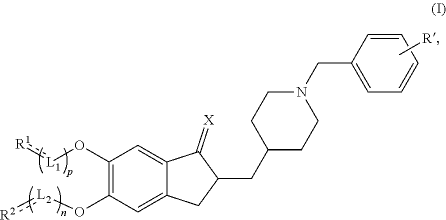

or a pharmaceutically acceptable salt and/or solvate thereof,
in which
   [symbol] represents a single or a double bond,
X is an oxygen atom or a group N—OH,
R¹ and R² are each independently H, an optionally substituted nitrogen-containing heterocyclyl group, an optionally substituted C₁-C₆ aliphatic chain or an optionally substituted aryl, wherein up to 4 methylene units of said aliphatic chain are optionally replaced by O, C(O), NH or N—C₁-C₆alkyl, provided that at least one of R¹ and R² is an optionally substituted nitrogen-containing heterocyclyl group,
L₁ and L₂ are each independently a divalent radical derived from a C₁-C₁₂ aliphatic chain, wherein one or more, preferably one to four, methylene unit(s) are optionally replaced by arylene, —O—, —S—, —C(═O)—, —SO₂— or —N(C₁-C₆ alkyl)-, wherein said aliphatic chain is optionally substituted,
p and n are each independently 0 or 1, provided that p is 1 when R¹ is an optionally substituted nitrogen-containing heterocyclyl group and n is 1 when R² is an optionally substituted nitrogen-containing heterocyclyl group, and
R′ is H, halogen, an optionally substituted C₁-C₆ aliphatic chain, an optionally substituted aryl, an optionally substituted heteroaryl or an optionally substituted C₁-C₆ alkyl-aryl, wherein up to 4 methylene units of said aliphatic chain are optionally replaced by O, C(O), NH or N—C₁-C₆alkyl.

According to a second aspect, the present invention relates to a pharmaceutical composition comprising at least one compound of formula (I) as defined above, a pharmaceutically acceptable salt and/or solvate thereof, and at least one pharmaceutically acceptable excipient.

According to a third aspect, the present invention also relates to the compound of formula (I), a pharmaceutically acceptable salt and/or solvate thereof, or the pharmaceutical composition according to the invention for use as a drug.

The present invention also relates to the use of a compound of formula (I) according to the invention, a pharmaceutically acceptable salt and/or solvate thereof, or the pharmaceutical composition according to the invention as a drug or for the manufacture of a drug.

The present invention also relates to a method for preventing and/or treating compulsive disorders, including addiction, eating disorders and obsessive-compulsive disorders, and Alzheimer's disease, comprising administering to a person in need thereof an effective dose of a compound of formula (I), a pharmaceutically acceptable salt and/or solvate thereof, or the pharmaceutical composition according to the invention.

### DETAILED DESCRIPTION

### Definitions

The term “stereoisomers” used in this invention refers to configurational stereoisomers and more particularly to optical isomers. Optical isomers that are not mirror images of one another are thus designated as “diastereoisomers”, and optical isomers, which are non-superimposable mirror images are designated as “enantiomers”. An equimolar mixture of two enantiomers of a chiral compound is designated as a racemic mixture or racemate.

For the purpose of the invention, the term “pharmaceutically acceptable” is intended to mean what is useful to the preparation of a pharmaceutical composition, and what is generally safe and non-toxic, for a pharmaceutical use.

The term “pharmaceutically acceptable salt and/or solvate” is intended to mean, in the framework of the present invention, a salt and/or solvate of a compound which is pharmaceutically acceptable, as defined above, and which possesses the pharmacological activity of the corresponding compound.

The pharmaceutically acceptable salts comprise:
(1) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L25 tartaric, tartaric, p-toluenesulfonic, trimethylacetic, and trifluoroacetic acid and the like, and
(2) base addition salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

Acceptable solvates for the therapeutic use of the compounds of the present invention include conventional solvates such as those formed during the last step of the preparation of the compounds of the invention due to the presence of solvents. As an example, mention may be made of solvates due to the presence of water (these solvates are also called hydrates) or ethanol.

The term “halogen”, as used in the present invention, refers to a fluorine, bromine, chlorine or iodine atom.

The term “Cr Cy aliphatic chain” designates a linear or branched hydrocarbon chain, completely saturated or containing one or more unsaturations, but not aromatic, comprising from x to y carbon atoms, notably from 1 to 12 carbon atoms, preferably from 1 to 6 carbon atoms. According to the present invention, the term “aliphatic chain” includes substituted or unsubstituted, linear or branched, alkyl, alkenyl or alkynyl groups.

The term “C₁-C₆ alkyl”, as used in the present invention, refers to a straight or branched monovalent saturated hydrocarbon chain containing from 1 to 6 carbon atoms including, but not limited to, methyl, ethyl, n-propyl, iso-propyl, n-butyl, iso-butyl, sec-butyl, t-butyl, n-pentyl, n-hexyl, and the like.

The term “C₂-C₆ alkenyl”, as used in the present invention, refers to a straight or branched monovalent unsaturated hydrocarbon chain containing from 2 to 6 carbon atoms and comprising at least one double bond including, but not limited to, ethenyl, propenyl, butenyl, pentenyl, hexenyl and the like.

The term “C₂-C₆ alkynyl”, as used in the present invention, refers to a straight or branched monovalent unsaturated hydrocarbon chain containing from 2 to 6 carbon atoms and comprising at least one triple bond including, but not limited to, ethynyl, propynyl, butynyl, pentynyl, hexynyl and the like.

The term “C₁-C₁₂ alkanediyl” as used in the present invention, refers to a straight or branched divalent saturated hydrocarbon chain containing from 1 to 12 carbon atoms including, but not limited to, methanediyl (methylene), ethanediyl, propanediyl, butanediyl, pentanediyl, hexanediyle, and the like.

The term “C₁-C₁₂ alkenediyl”, as used in the present invention, refers to a straight or branched divalent unsaturated hydrocarbon chain containing from 1 to 12 carbon atoms and comprising at least one double bond including, but not limited to, ethenediyl, propenediyl, butenediyl, pentenediyl, hexenediyl and the like.

The term “aryl group”, as used in the present invention, refers to an aromatic hydrocarbon group preferably comprising from 6 to 12 carbon atoms and comprising one or more fused rings, such as, for example but not limited to, a phenyl or naphthyl group. Advantageously, it is a phenyl group.

The term “heterocyclyl group” as used in the present invention, refers to an aromatic or non-aromatic, saturated or unsaturated, monocyclic or polycyclicgroup (comprising fused, bridged or spiro rings) comprising preferably 5 to 10, notably 5, 6, 9 or 10 atoms in the ring(s), in which one or several, notably one to four, advantageously one or two, atom(s) of the ring(s) carbon atoms each is replaced with heteroatoms selected from a sulfur atom, an oxygen atom and a nitrogen atom. When the heterocyclyl group is a “nitrogen-containing heterocyclyl group”, it is meant that at least one the heteroatoms in the ring(s) is a nitrogen atom. It is not excluded that a “nitrogen-containing heterocyclyl group” according to the invention comprises oxygen atom(s) and/or sulfur atom(s) in the ring(s), in addition to the at least one nitrogen atom. The nitrogen-containing heterocyclyl group according to the invention may be under its cationic form when it is linked to the rest of the molecule via a nitrogen atom, thus having a valence of 4.

When said heterocyclyl group is an aromatic compound, it may be referred to as a “heteroaryl” in the present disclosure. Examples of “nitrogen-containing heteroaryl” includes, without being limited to, pyrrolyl, pyridyl, thiazinyl, thiazolyl, isothiazolyl, imidazolyl, pyrazolyl, triazolyl, tetrazolyl, pyridazinyl, purinyl, pyrimidinyl, pyrazinyl, triazinyl, quinolinyl, isoquinolinyl, isoindolyl or indolyl. Preferably, in the context of the present invention, a nitrogen-containing heteroaryl is not a pyridyl.

When said heterocyclyl group is a non aromatic compound, it may be referred to as a “heterocycloalkyl” in the present disclosure. Examples of “nitrogen-containing heterocycloalkyl” include, without being limited to piperidinyl, piperizinyl, pyrrolidinyl, pyrazolidinyl, imidazolidinyl, thiazolidinyl, isothiazolidinyl, indolinyl and isoindolinyl.

The term “C₁-C₆-alkyl aryl” as used in the present invention, refers to an alkyl group as defined above substituted by an aryl as defined above. Advantageously, a “C₁-C₆-alkyl aryl” is a benzyl group.

In the context of the present invention, an “optionally substituted” group means that the group in question is optionally substituted with one or more, preferably one or two, substituents which may be selected in particular from halogen, C₁-C₆ alkyl, C₁-C₆ haloalkyl, C₂-C₆ alkene, C₂-C₆ alkyne, aryl, N₃, oxo, NR^{a}R^{b}, COR^{c}, CO₂R^{d}, CONR^{e}R^{f}, OR^{g}, N⁺R^{h}RⁱR^{j}, CN and NO₂ wherein R^{a} to R^{j} are, independently of one another, H, C₁-C₆ alkyl or aryl, preferably H or C₁-C₆ alkyl. Advantageously, an “optionally substituted” group is substituted with one or two substituents selected from the group consisting of C₁-C₆ alkyl and OR^{g} wherein R^{g} is H or C₁-C₆ alkyl.

The term “C₁-C₆ haloalkyl” refers to a C₁-C₆ alkyl chain as defined above wherein one or more hydrogen atoms are replaced by a halogen atom selected from fluorine, chlorine, bromine or iodine, preferably a fluorine atom. For example, it is a CF₃ group.

In the context of the present invention, “unsaturated” means that the hydrocarbon chain may contain one or more unsaturation(s), i.e. a double bond C═C or a triple bond C≡C, advantageously one.

The term “leaving group” refers to an atom or group of atoms that are easily able to break away from a molecule with a lone pair, breaking the bond between it and the molecule.

The term “pharmaceutical composition” in the framework of the present invention is understood as a composition having preventive and curative properties.

The term “prodrug” relates to a typically pharmacologically inactive or less active derivative of an active drug that undergoes in cellulo or in vivo biotransformation to release the active drug by chemical or enzymatic reactions. By “pharmacologically inactive or less active derivative”, it is understood, in the context of the invention, that the prodrug does not have relevant activity with respect to the inhibition of the acetylcholinesterase active site in an in vitro setting. However, the oxidized form is active in in cellulo and in vivo assays because such conditions allow the transformations required to provide the active drug. Prodrugs can offer many advantages over parent drugs such as increased brain penetration, and so an increased activity in the brain, enhanced stability and improved bioavailability.

In the context of the invention, the prodrug compounds as defined in the present disclosure possess increased brain activity. After brain penetration, the prodrug compounds are oxidized to provide the corresponding active drugs, i.e. the potent acetylcholinesterase inhibitors.

### Compounds of Formula (I)

The compound of formula (I) for use according to the present invention can be in the form of a stereoisomer or a mixture of stereoisomers, such as a mixture of enantiomers, diastereoisomers or tautomers, notably a racemic mixture.

In the context of the present invention, the nitrogen atoms of the compound of formula (I) may have a valence of 3 or a valence of 4. When they have a valence of 4, for example when being protonated, the nitrogen atoms are under cationic form.

In particular, referring to R¹ and/or R², the nitrogen containing heterocyclyl group may be linked to the rest of the molecule via a carbon atom or a nitrogen atom. When it is linked to the rest of the molecule via a nitrogen atom, said nitrogen atom may have a valence of 3 or a valence of 4. When said nitrogen atom has a valence of 4, it is under its cationic form, i.e. an ammonium. It is notably the case when the heterocyclyl group is a heteroaryl.

In particular, the nitrogen of the piperidine moiety that bears the benzyl group in the compound of formula (I) may have a valence of 3 or a valence of 4. When said nitrogen atom has a valence of 4, it is under its cationic form, i.e. an ammonium.

In the context of the present invention, the nitrogen-containing heterocyclyl group, referring to R¹ and/or R², typically do not contain any other heteroatom than nitrogen atom(s).

In the context of the present invention, the nitrogen-containing heterocyclyl group, referring to R¹ and/or R², is selected from the group consisting of optionally substituted pyrrolyl, pyridyl, thiazinyl, thiazolyl, isothiazolyl, imidazolyl, pyrazolyl, triazolyl, tetrazolyl, pyridazinyl, purinyl, pyrimidinyl, pyrazinyl, triazinyl, quinolinyl, isoquinolinyl, isoindolyl, indolyl, piperidinyl, piperizinyl, pyrrolidinyl, pyrazolidinyl, imidazolidinyl, thiazolidinyl, isothiazolidinyl, dihydroquinolinyl, dihydroisoquinolinyl, tetrahydroquinolinyl, tetrahydroisoquinolinyl, indolinyl and isoindolinyl.

According to some embodiments, R¹ and R² are each independently an optionally substituted nitrogen-containing heterocyclyl group or an optionally substituted C₁-C₆ aliphatic chain, wherein up to 4 methylene units of said aliphatic chain are optionally replaced by 0, C(O), NH or N—C₁-C₆alkyl, provided that at least one of R¹ and R² is an optionally substituted nitrogen-containing heterocyclyl group.

According to some embodiments, R¹ and R² are each independently an optionally substituted nitrogen-containing heterocyclyl group as defined below, identical or different.

According to preferred embodiments, R¹ and R² are different, one of R¹ and R² is an optionally substituted nitrogen-containing heterocyclyl group as defined below and the other is H, an optionally substituted C₁-C₆ aliphatic chain or an optionally substituted aryl, preferably H, an optionally substituted C₁-C₆ aliphatic chain, such as C₁-C₆ alkyl or an optionally substituted aryl, such as phenyl, more preferably, H, a C₁-C₆ alkyl or an aryl, such as phenyl, even more preferably, a C₁-C₆ alkyl including a methyl, an ethyl, a propyl, a tert-butyl, a n-butyl, notably a methyl.

According to a preferred embodiment, R¹ and/or R², preferably one of R¹ and R² is an optionally substituted nitrogen-containing heterocyclyl group having from 1 to 4 nitrogen atoms replacing carbon atom in the ring, preferably 1 or 2, more preferably 1.

According to a preferred embodiment, R¹ and/or R², preferably one of R¹ and R² is an optionally substituted nitrogen-containing heterocyclyl group selected from the group consisting of optionally substituted pyrrolyl, pyridyl, thiazinyl, thiazolyl, isothiazolyl, imidazolyl, pyrazolyl, triazolyl, tetrazolyl, pyridazinyl, purinyl, pyrimidinyl, pyrazinyl, triazinyl, quinolinyl, isoquinolinyl, isoindolyl, indolyl, piperidinyl, piperizinyl, pyrrolidinyl, pyrazolidinyl, imidazolidinyl, thiazolidinyl, isothiazolidinyl, dihydroquinolinyl, dihydroisoquinolinyl, tetrahydroquinolinyl, tetrahydroisoquinolinyl, indolinyl and isoindolinyl.

In a particular embodiment, R¹ and/or R², preferably one of R¹ and R² is an optionally substituted nitrogen-containing heteroaryl, notably selected from the group consisting of pyrrolyl, thiazinyl, thiazolyl, isothiazolyl, imidazolyl, pyrazolyl, triazolyl, tetrazolyl, pyridazinyl, purinyl, pyrimidinyl, pyrazinyl, triazinyl, quinolinyl, isoquinolinyl, isoindolyl or indolyl.

In another particular embodiment, R¹ and/or R², preferably one of R¹ and R² is an optionally substituted nitrogen-containing heterocycloalkyl, notably selected from the group consisting of piperidinyl, piperizinyl, pyrrolidinyl, pyrazolidinyl, imidazolidinyl, thiazolidinyl, isothiazolidinyl, dihydroquinolinyl, dihydroisoquinolinyl, tetrahydroquinolinyl, tetrahydroisoquinolinyl, indolinyl and isoindolinyl.

Preferably, R¹ and/or R², when being a nitrogen-containing heterocyclyl group is unsubstituted or substituted with one or more substituents, preferably one or two, more preferably one, selected from the group consisting in halogen, C₁-C₆ alkyl, C₁-C₆ haloalkyl, C₂-C₆ alkene, C₂-C₆ alkyne, aryl, N₃, oxo, NH₂, NH—C₁-C₆alkyl, N(C₁-C₆alkyl)₂, C(O)H, CO₂—C₁-C₆alkyl, CO₂H, CN and NO₂, preferably halogen, such as Cl, OH, NH₂, C₁-C₆ alkyl, such as a methyl, and O—C₁-C₆ alkyl, such as a methoxy.

Preferably, R¹ and/or R², preferably one of R¹ and R², is a group corresponding to one of the following formula:

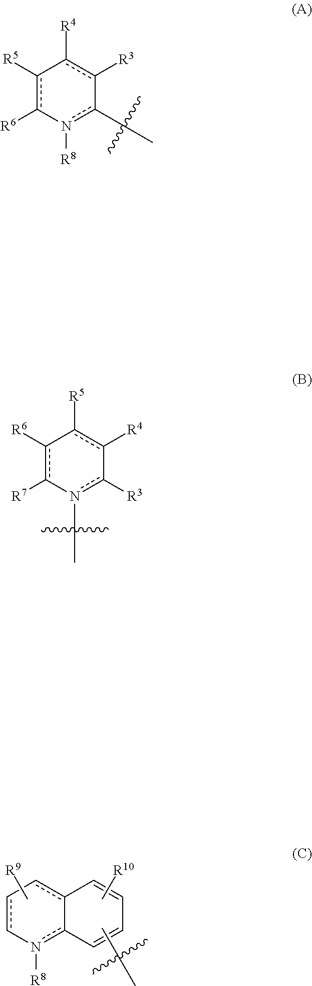

in which
R³ to R⁸ are each independently selected in the group consisting of H, halogen, OH, NH₂, NH—C₁-C₆ alkyl, a C₁-C₆ aliphatic chain, aryl, heteroaryl and C₁-C₆ alkyl-aryl, wherein up to 4 methylene units of said aliphatic chain are optionally replaced by 0, C(O), NH or N—C₁-C₆alkyl, said aliphatic chain, aryl, heteroaryl or alkyl-aryl being optionally substituted, or one or more among the couples R³-R⁴, R⁴-R⁵, R⁵-R⁶ and R⁶-R⁷ form together with the carbon atoms to which they are bonded a 5 or 6 membered aromatic or non aromatic optionally substituted ring,
R⁹ and R¹⁰ are each independently one or more substituents selected in the group consisting of H, a C₁-C₆ aliphatic chain, aryl, heteroaryl and C₁-C₆ alkyl-aryl, wherein up to 4 methylene units of said aliphatic chain are optionally replaced by O, C(O), NH or N—C₁-C₆alkyl, said aliphatic chain, aryl, heteroaryl or alkyl-aryl being optionally substituted,
   [symbol] represents a single or a double bond, and
   [symbol] represents the bond between R¹ and the rest of the molecule.

It is understood that, in formula (C), R⁹ is one or more substituent(s) bear by the carbon atom(s) of the nitrogen-containing ring and R¹⁰ is one or more substituent(s) bear by the carbon atom(s) of the phenyl moiety linked to the rest of the molecule.

More preferably, R³ to R⁸ are each independently selected in the group consisting of H, halogen, OH, NH₂, NH—C₁-C₆ alkyl, a C₁-C₆ alkyl and aryl, such as phenyl, or
one or more among the couples R³-R⁴, R⁴-R⁵, R⁵-R⁶ and R⁶-R⁷ form together with the carbon atoms to which they are bonded a 5 or 6-membered aromatic or non aromatic ring, unsubstituted or substituted with one or more, preferably one, substituent selected from halogen, OH, NH₂, NH—C₁-C₆ alkyl, C₁-C₆ alkyl and O—C₁-C₆ alkyl.

According to preferred embodiments, R⁸ is H or C₁-C₆ alkyl, such as methyl, ethyl or isopropyl, notably methyl.

According to preferred embodiments, one among the couples R³-R⁴, R⁴-R⁵, R⁵-R⁶ and R⁶-R⁷ form together with the carbon atoms to which they are bonded a 6-membered aromatic ring, such as a phenyl, unsubstituted or substituted with one or more, preferably one, substituent selected from halogen, OH, NH₂, NH—C₁-C₆alkyl, C₁-C₆ alkyl and O—C₁-C₆ alkyl, such as methoxy, the other radicals among R³ to R⁷ being H.

According to preferred embodiments, R⁹ and R¹⁰ are each independently one or more substituents, preferably one, selected in the group consisting of H, a C₁-C₆ alkyl, halogen, OH, NH₂ and NH—C₁-C₆alkyl. More preferably, R⁹ is one substituent selected in the group consisting of OH and halogen, such as Cl, and R¹⁰ is H.

When R¹ or R² is of formula (B) as defined above, the nitrogen to which the rest of the molecule is linked may be neutral or cationic, depending on the bonds that said nitrogen forms in the ring, said bonds being single or double, provided that the valence of the nitrogen is never higher than 4.

In a particular embodiment, R¹ and/or R², preferably one of R¹ and R², is an optionally substituted 8 to 10-membered bicyclic nitrogen-containing heterocyclyl group, notably selected from the group consisting of quinolinyl, isoquinolinyl, dihydroquinolinyl, dihydroisoquinolinyl, unsubstituted or substituted with one or more, preferably one, substituent selected from halogen, OH, NH₂, NH—C₁-C₆ alkyl, C₁-C₆ alkyl or O—C₁-C₆ alkyl, such as methoxy.

The formula (I) as defined herein encompasses both active compounds, i.e drugs, and prodrugs thereof.

According to a particular embodiment, when the compound of formula (I) is a prodrug, R¹ and/or R², preferably one of R¹ and R², is an optionally substituted nitrogen-containing heterocycloalkyl, meaning that the ring is non aromatic. In such an embodiment, if the other R¹ or R² is also an optionally substituted nitrogen-containing heterocyclyl group, it is preferably a nitrogen-containing heterocycloalkyl, identical or different. According to such an embodiment, the heterocycloalkyl may undergo an oxidation reaction once the prodrug has penetrated into the brain to provide the active drug.

Therefore, according to a specific embodiment, when compound of formula (I) is a drug, R¹ or R², when being nitrogen-containing heterocyclyl group, is an optionally substituted nitrogen-containing heteroaryl, meaning that the ring is aromatic. In such an embodiment, if the other R¹ or R² is also an optionally substituted nitrogen-containing heterocyclyl group, it is preferably a nitrogen-containing heterocycloaryl, identical or different.

Accordingly, in a prodrug according to the invention, the optionally substituted nitrogen-containing heterocycloalkyl, referring to R¹ and/or R², is preferably selected from the group consisting in pyrrolyl, thiazinyl, thiazolyl, isothiazolyl, imidazolyl, pyrazolyl, triazolyl, tetrazolyl, pyridazinyl, purinyl, pyrimidinyl, pyrazinyl, triazinyl, quinolinyl, isoquinolinyl, isoindolyl and indolyl, preferably quinolinyl and isoquinolinyl.

In a drug according to the invention, the optionally substituted nitrogen-containing heteroaryl, referring to R¹ and/or R², is preferably selected from the group consisting in piperidinyl, piperizinyl, pyrrolidinyl, pyrazolidinyl, imidazolidinyl, thiazolidinyl, isothiazolidinyl, dihydroquinolinyl, dihydroisoquinolinyl, tetrahydroquinolinyl, tetrahydroisoquinolinyl, indolinyl and isoindolinyl, preferably dihydroquinolinyl, dihydroisoquinolinyl, tetrahydroquinolinyl and tetrahydroisoquinolinyl.

In the context of the present invention, the drug compound is the oxidized form of the corresponding prodrug compound. For example, if the prodrug contains a heterocycloalkyl, it is oxidized and thus aromatized in the corresponding drug, thus giving the corresponding heteroaryl.

X is preferably an oxygen atom.

L₁ and L₂ are each independently linkers corresponding to a divalent radical derived from a C₁-C₁₂ aliphatic chain, wherein one or more, preferably one to four, more preferably one or two, methylene unit(s) are optionally replaced by arylene, —O—, —S—, —C(═O)—, —SO₂— or —N(C₁-C₆ alkyl)-, wherein said aliphatic chain is optionally substituted with one or more, preferably one, substituent selected from OH, halogen, C₁-C₆ alkyl or aryl, preferably OH or C₁-C₆ alkyl.

According to a preferred embodiment, L₁ and L₂ are each independently a divalent radical derived from an unsubstituted C₁-C₁₂ aliphatic chain, preferably a C₁-C₁₂ alkanediyl, a C₂-C₁₂ alkenediyl or a linker of formula —CH═, notably a C₁-C₆ alkanediyl or a linker of formula —CH═. More preferably, L₁ and L₂ are each independently a methylene group or a linker of formula —CH═. It is understood that when the linker is of formula —CH═, it is directly linked to R¹ via the double bond and to the oxygen atom via the single bond.

In the compound of formula (I), n and p are each independently 0 or 1, provided that p is 1 when R¹ is an optionally substituted nitrogen-containing heterocyclyl group and n is 1 when R² is an optionally substituted nitrogen-containing heterocyclyl group. p may thus be 0 or 1 when R¹ is not an optionally substituted nitrogen-containing heterocyclyl group and n may thus be 0 or 1 when R² is not an optionally substituted nitrogen-containing heterocyclyl group. Preferably, p is 0 when R¹ is not an optionally substituted nitrogen-containing heterocyclyl group and n is 0 when R² is not an optionally substituted nitrogen-containing heterocyclyl group. In other terms, when R¹ or R² is not an optionally substituted nitrogen-containing heterocyclyl group, said R¹ or R² is preferably directly linked to the oxygen atom bear by the indanone group, and not via a linker L₁ or L₂.

According to preferred embodiment, R′ is H, halogen, C₁-C₆ alkyl, aryl, heteroaryl or C₁-C₆ alkyl-aryl, said alkyl, aryl, heteroaryl or alkyl-aryl being unsubstituted or substituted with one or more, preferably one, substituent selected from OH, halogen, C₁-C₆ alkyl or aryl, notably OH or C₁-C₆ alkyl. Preferably, R′ is H, halogen or a C₁-C₆ alkyl, such as methyl or ethyl. More preferably, R′ is H.

In a particular embodiment, R¹ is an optionally substituted nitrogen-containing heterocyclyl group as defined in the present disclosure, R′ is H or a C₁-C₆ alkyl, such as methyl or ethyl, p is 1, n is 0 and R² is a C₁-C₆ alkyl including a methyl, an ethyl, a propyl, a tert-butyl, a n-butyl. Preferably, R¹ is an optionally substituted nitrogen-containing heterocyclyl group as defined in the present disclosure, p is 1, L₁ is a divalent radical derived from a C₁-C₁₂ aliphatic chain, preferably a methylene or a linker of formula —CH═, R² is methyl, n is 0, and R′ is H.

According to specific embodiments, in the compound of formula (I), R¹ is selected from the group consisting of quinolinyl, isoquinolinyl, dihydroquinolinyl, dihydroisoquinolinyl, unsubstituted or substituted with one or more, preferably one, substituent selected from halogen, OH, NH₂, NH—C₁-C₆alkyl, C₁-C₆ alkyl or O—C₁-C₆ alkyl, such as methoxy, R² is methyl, n is 0, p is 1, L₁ is a divalent radical derived from a C₁-C₁₂ aliphatic chain, preferably a methylene or a linker of formula —CH═; and R′ is H.

In other embodiment, R² is an optionally substituted nitrogen-containing heterocyclyl group as defined in the present disclosure, R′ is H or a C₁-C₆ alkyl, such as methyl or ethyl, p is 0, n is 1 and R¹ is a C₁-C₆ alkyl including a methyl, an ethyl, a propyl, a tert-butyl, a n-butyl. Preferably, R² is an optionally substituted nitrogen-containing heterocyclyl group as defined in the present disclosure, p is 0, L₂ is a divalent radical derived from a C₁-C₁₂ aliphatic chain, preferably a methylene or a linker of formula —CH═, R¹ is methyl, n is 1, and R′ is H.

In other embodiments, in the compound of formula (I), R² is selected from the group consisting of quinolinyl, isoquinolinyl, dihydroquinolinyl, dihydroisoquinolinyl, unsubstituted or substituted with one or more, preferably one, substituent selected from halogen, OH, NH₂, NH—C₁-C₆alkyl, C₁-C₆ alkyl or O—C₁-C₆ alkyl, such as methoxy, R¹ is methyl, n is 1, p is 0, L₂ is a divalent radical derived from a C₁-C₁₂ aliphatic chain, preferably a methylene or a linker of formula —CH═; and R′ is H.

According to a preferred embodiment, the compound of formula (I) is selected in the group consisting of:

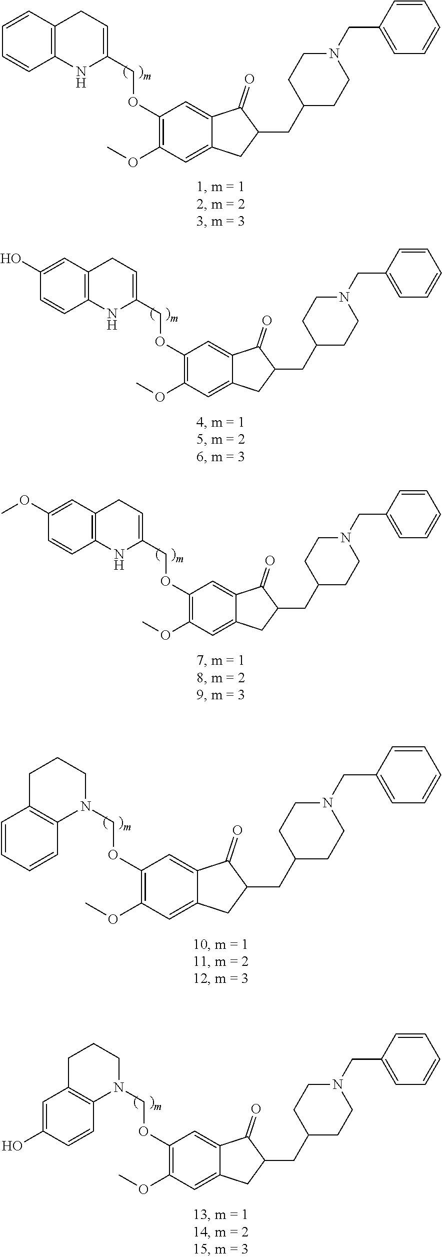

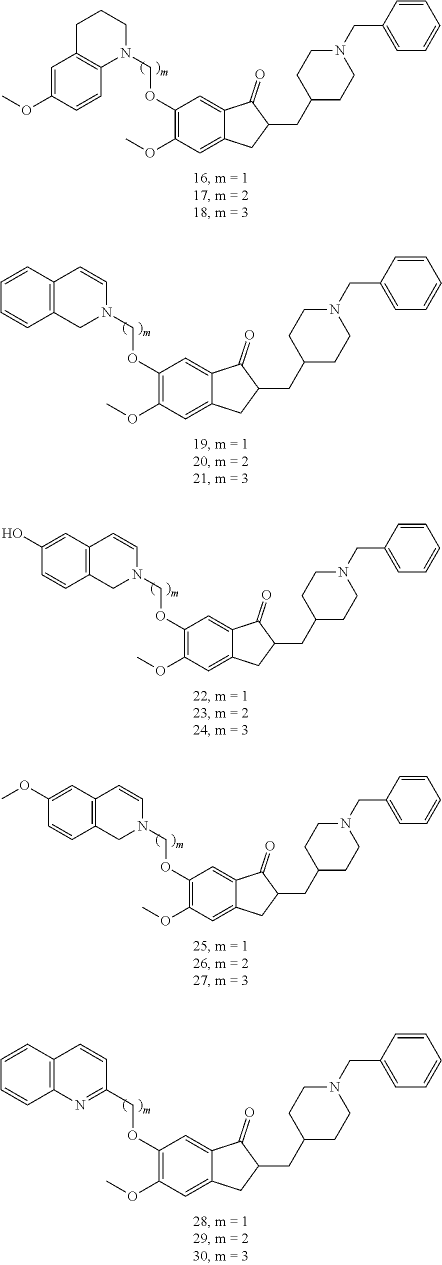

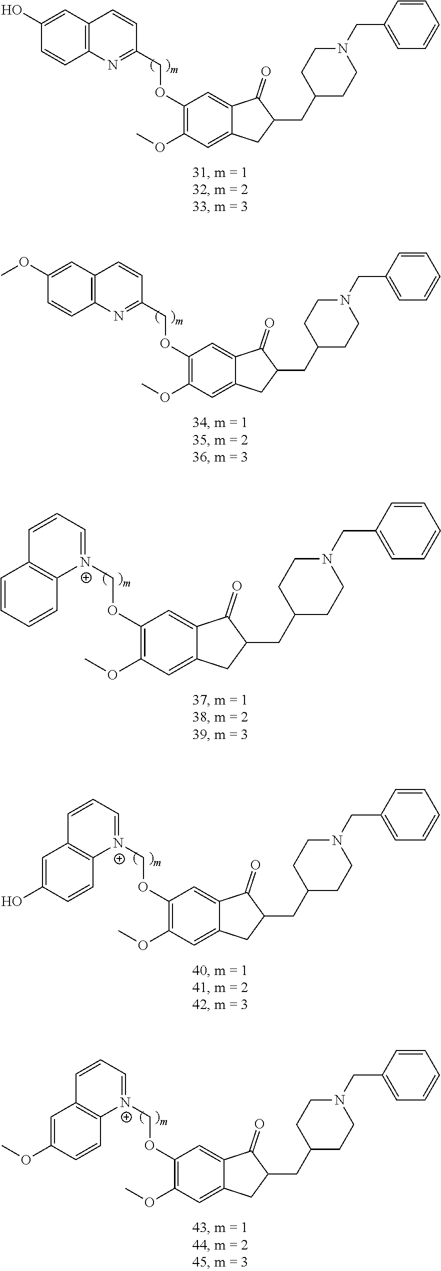

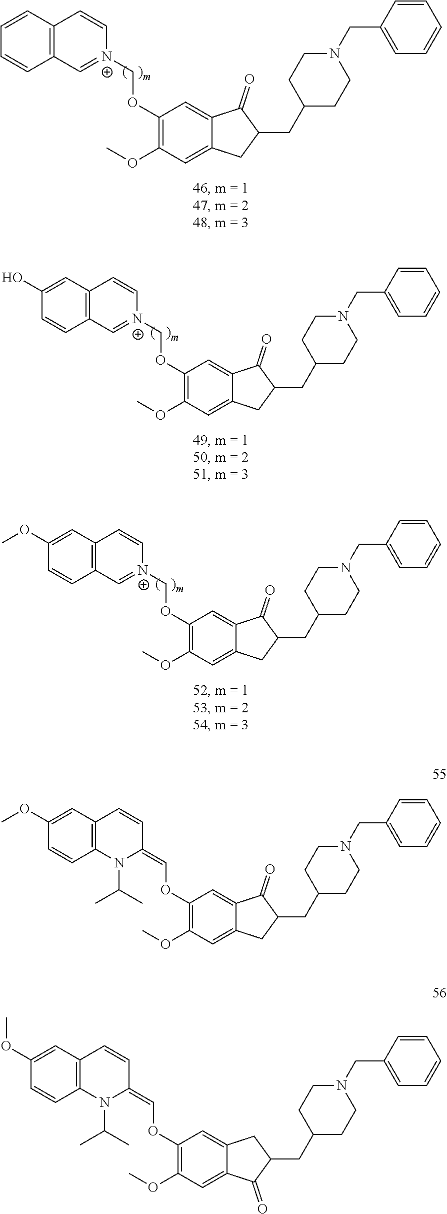

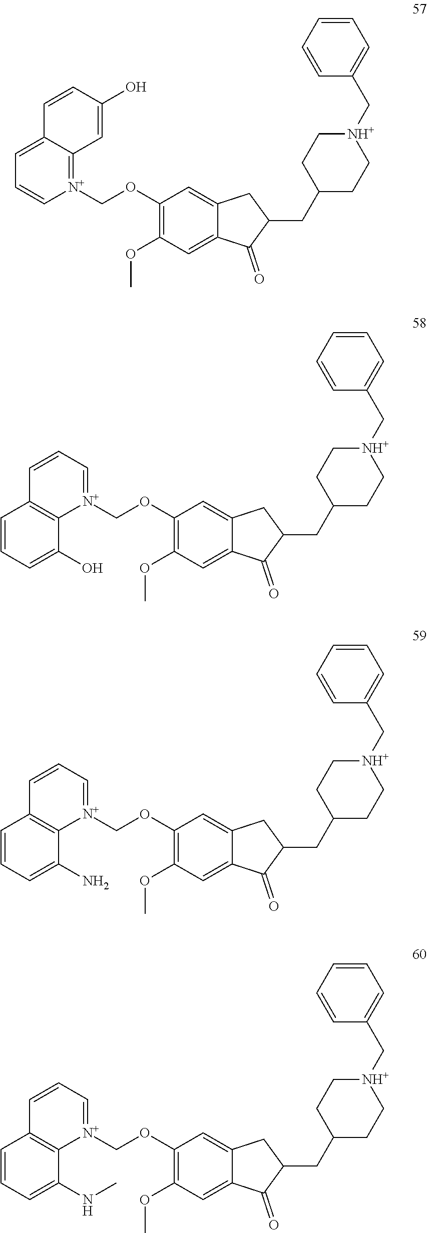 [SMILES: COc1cc2c(cc1OC[n+]1cccc3ccc(O)cc31)CC(CC1CC[NH+](Cc3ccccc3)CC1)C2=O ; COc1cc2c(cc1OC[n+]1cccc3cccc(O)c31)CC(CC1CC[NH+](Cc3ccccc3)CC1)C2=O ; COc1cc2c(cc1OC[n+]1cccc3cccc(N)c31)CC(CC1CC[NH+](Cc3ccccc3)CC1)C2=O ; CNc1cccc2ccc[n+](COc3cc4c(cc3OC)C(=O)C(CC3CC[NH+](Cc5ccccc5)CC3)C4)c12]

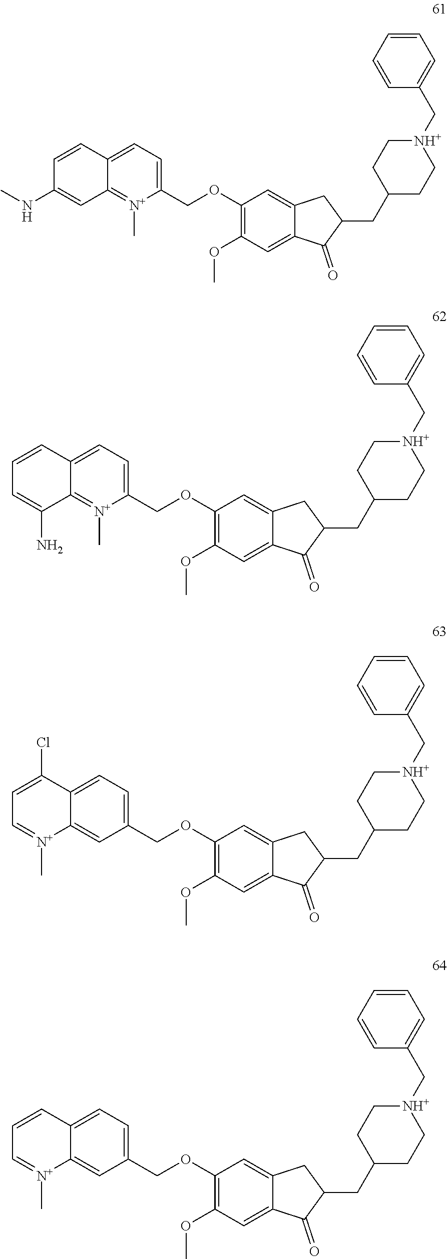 [SMILES: CNc1ccc2ccc(COc3cc4c(cc3OC)C(=O)C(CC3CC[NH+](Cc5ccccc5)CC3)C4)[n+](C)c2c1 ; COc1cc2c(cc1OCc1ccc3cccc(N)c3[n+]1C)CC(CC1CC[NH+](Cc3ccccc3)CC1)C2=O ; COc1cc2c(cc1OCc1ccc3c(Cl)cc[n+](C)c3c1)CC(CC1CC[NH+](Cc3ccccc3)CC1)C2=O ; COc1cc2c(cc1OCc1ccc3ccc[n+](C)c3c1)CC(CC1CC[NH+](Cc3ccccc3)CC1)C2=O]

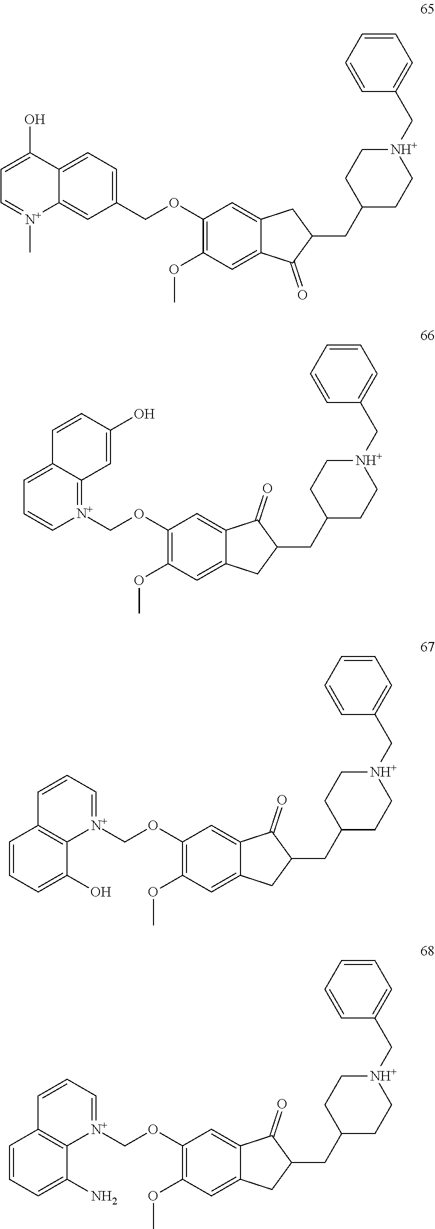 [SMILES: COc1cc2c(cc1OCc1ccc3c(O)cc[n+](C)c3c1)CC(CC1CC[NH+](Cc3ccccc3)CC1)C2=O ; COc1cc2c(cc1OC[n+]1cccc3ccc(O)cc31)C(=O)C(CC1CC[NH+](Cc3ccccc3)CC1)C2 ; COc1cc2c(cc1OC[n+]1cccc3cccc(O)c31)C(=O)C(CC1CC[NH+](Cc3ccccc3)CC1)C2 ; COc1cc2c(cc1OC[n+]1cccc3cccc(N)c31)C(=O)C(CC1CC[NH+](Cc3ccccc3)CC1)C2]

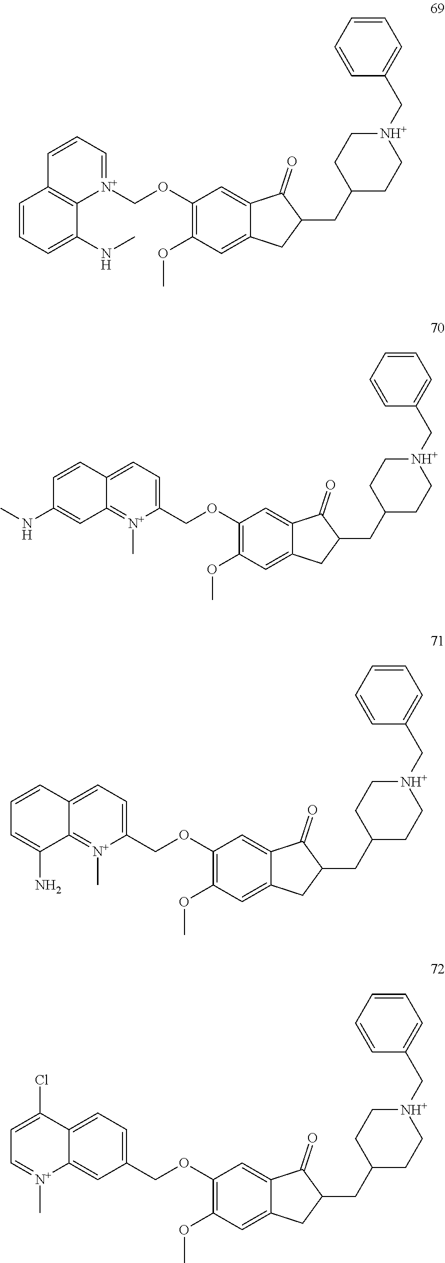 [SMILES: CNc1cccc2ccc[n+](COc3cc4c(cc3OC)CC(CC3CC[NH+](Cc5ccccc5)CC3)C4=O)c12 ; CNc1ccc2ccc(COc3cc4c(cc3OC)CC(CC3CC[NH+](Cc5ccccc5)CC3)C4=O)[n+](C)c2c1 ; COc1cc2c(cc1OCc1ccc3cccc(N)c3[n+]1C)C(=O)C(CC1CC[NH+](Cc3ccccc3)CC1)C2 ; COc1cc2c(cc1OCc1ccc3c(Cl)cc[n+](C)c3c1)C(=O)C(CC1CC[NH+](Cc3ccccc3)CC1)C2]

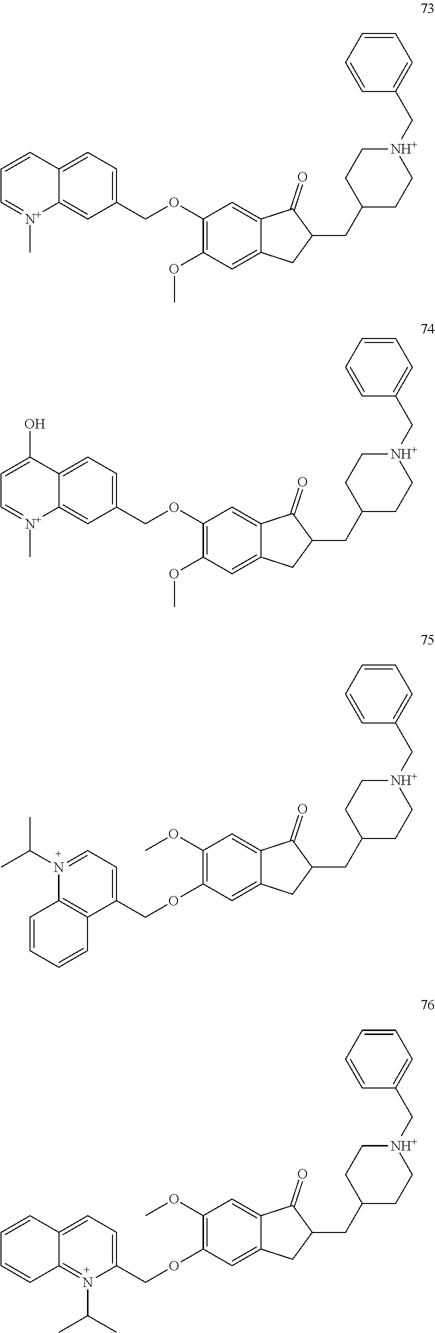

In some embodiments, the compound of formula (I) is selected in the group consisting of compounds 1′ to 76′ in which X is N—OH and the rest of the substituents are respectively as defined in compounds 1 to 76 hereabove. For example, in compound 1′, X is N—OH, and the other substituents are as in compound 1, i.e. R′ is H, R² is CH₃, L₁ is —CH₂—, p is 1, n is 0 and R¹ is dihydroquinolinyl.

In a particular embodiment, the compound of formula (I) according to the invention is a prodrug as defined above, notably selected in the group consisting of:

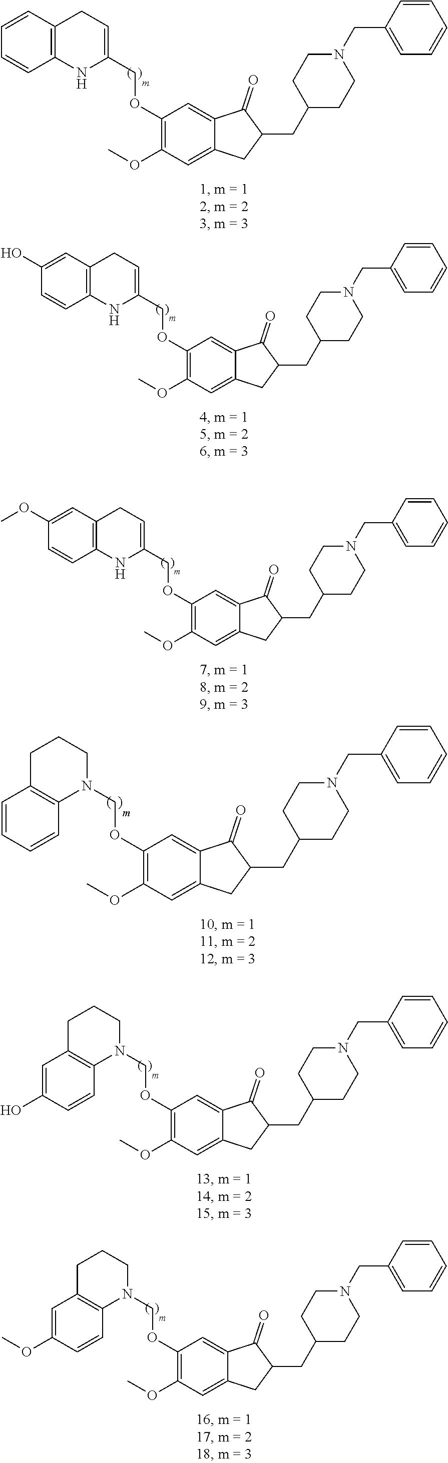 [SMILES: COc1cc2c(cc1OCC1=CCc3ccccc3N1)C(=O)C(CC1CCN(Cc3ccccc3)CC1)C2 ; COc1cc2c(cc1OCC1=CCc3cc(O)ccc3N1)C(=O)C(CC1CCN(Cc3ccccc3)CC1)C2 ; COc1ccc2c(c1)CC=C(COc1cc3c(cc1OC)CC(CC1CCN(Cc4ccccc4)CC1)C3=O)N2 ; COc1cc2c(cc1OCN1CCCc3ccccc31)C(=O)C(CC1CCN(Cc3ccccc3)CC1)C2 ; COc1cc2c(cc1OCN1CCCc3cc(O)ccc31)C(=O)C(CC1CCN(Cc3ccccc3)CC1)C2 ; COc1ccc2c(c1)CCCN2COc1cc2c(cc1OC)CC(CC1CCN(Cc3ccccc3)CC1)C2=O]

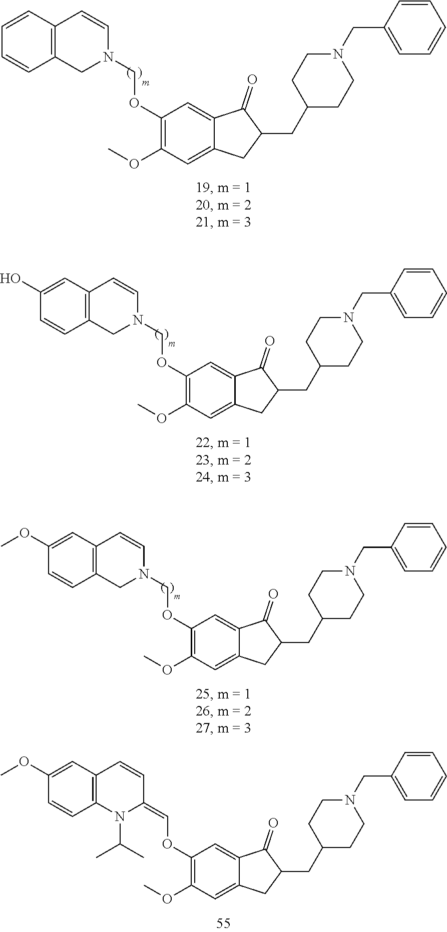 [SMILES: COc1cc2c(cc1OCN1C=Cc3ccccc3C1)C(=O)C(CC1CCN(Cc3ccccc3)CC1)C2 ; COc1cc2c(cc1OCN1C=Cc3cc(O)ccc3C1)C(=O)C(CC1CCN(Cc3ccccc3)CC1)C2 ; COc1ccc2c(c1)C=CN(COc1cc3c(cc1OC)CC(CC1CCN(Cc4ccccc4)CC1)C3=O)C2 ; COc1ccc2c(c1)C=C/C(=C/Oc1cc3c(cc1OC)CC(CC1CCN(Cc4ccccc4)CC1)C3=O)N2C(C)C]

In a particular embodiment, the compound of formula (I) according to the invention is a drug as defined above, notably selected in the group consisting of:

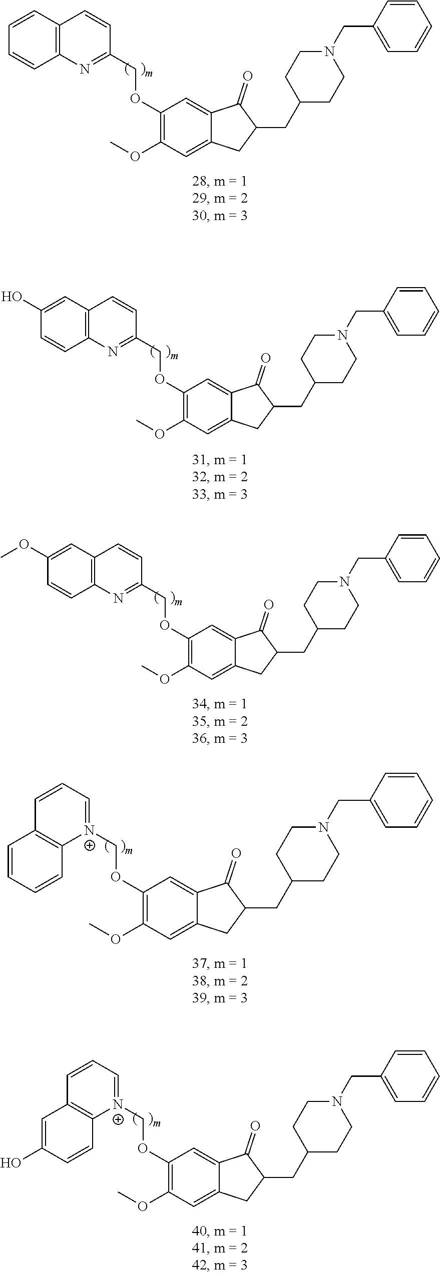

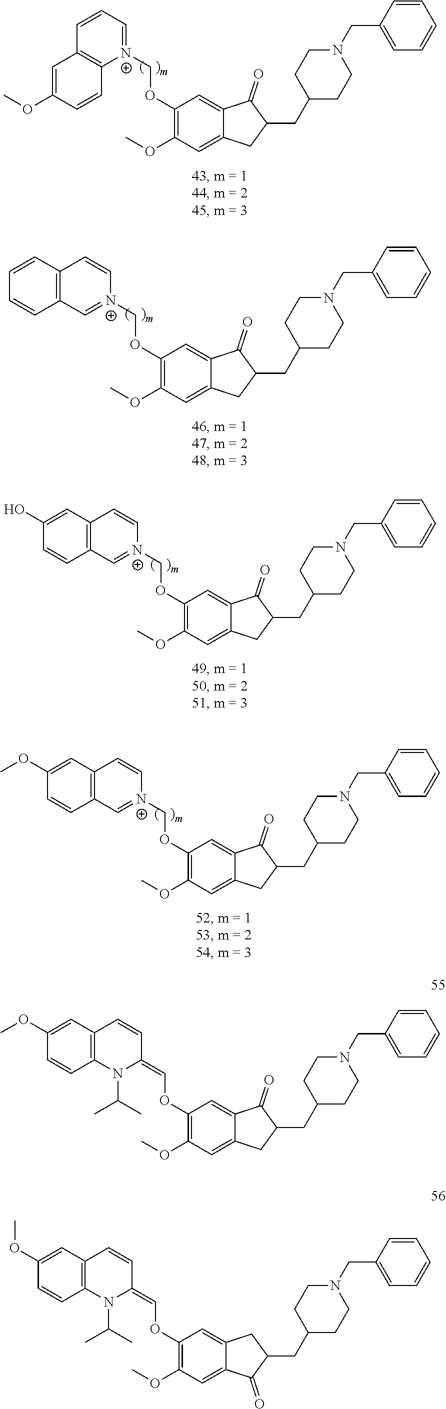

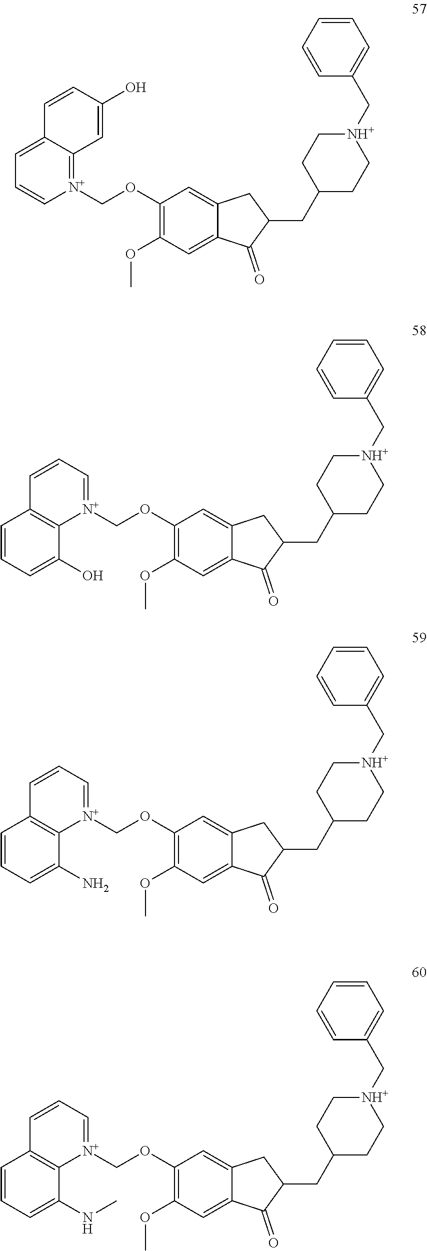 [SMILES: COc1cc2c(cc1OC[n+]1cccc3ccc(O)cc31)CC(CC1CC[NH+](Cc3ccccc3)CC1)C2=O ; COc1cc2c(cc1OC[n+]1cccc3cccc(O)c31)CC(CC1CC[NH+](Cc3ccccc3)CC1)C2=O ; COc1cc2c(cc1OC[n+]1cccc3cccc(N)c31)CC(CC1CC[NH+](Cc3ccccc3)CC1)C2=O ; CNc1cccc2ccc[n+](COc3cc4c(cc3OC)C(=O)C(CC3CC[NH+](Cc5ccccc5)CC3)C4)c12]

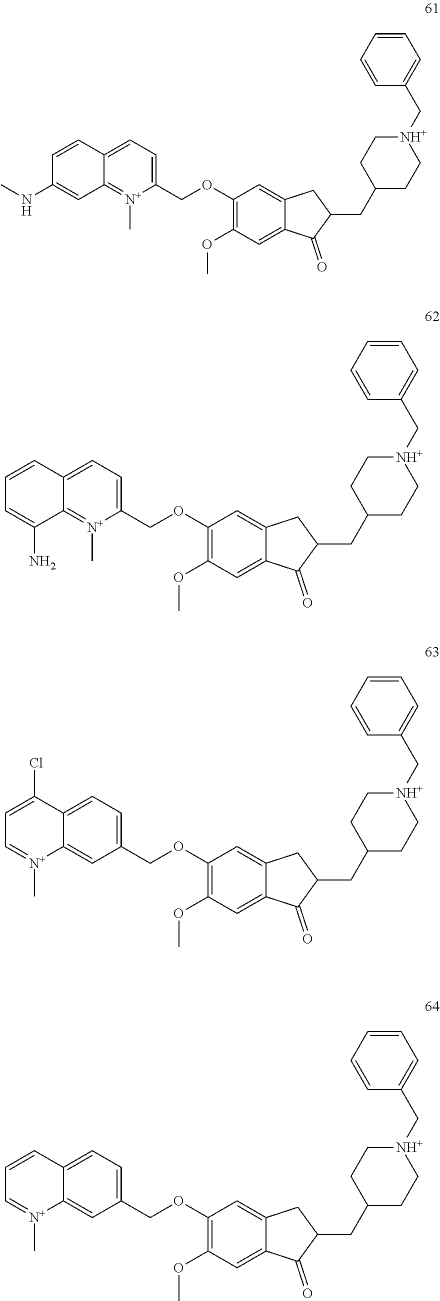 [SMILES: CNc1ccc2ccc(COc3cc4c(cc3OC)C(=O)C(CC3CC[NH+](Cc5ccccc5)CC3)C4)[n+](C)c2c1 ; COc1cc2c(cc1OCc1ccc3cccc(N)c3[n+]1C)CC(CC1CC[NH+](Cc3ccccc3)CC1)C2=O ; COc1cc2c(cc1OCc1ccc3c(Cl)cc[n+](C)c3c1)CC(CC1CC[NH+](Cc3ccccc3)CC1)C2=O ; COc1cc2c(cc1OCc1ccc3ccc[n+](C)c3c1)CC(CC1CC[NH+](Cc3ccccc3)CC1)C2=O]

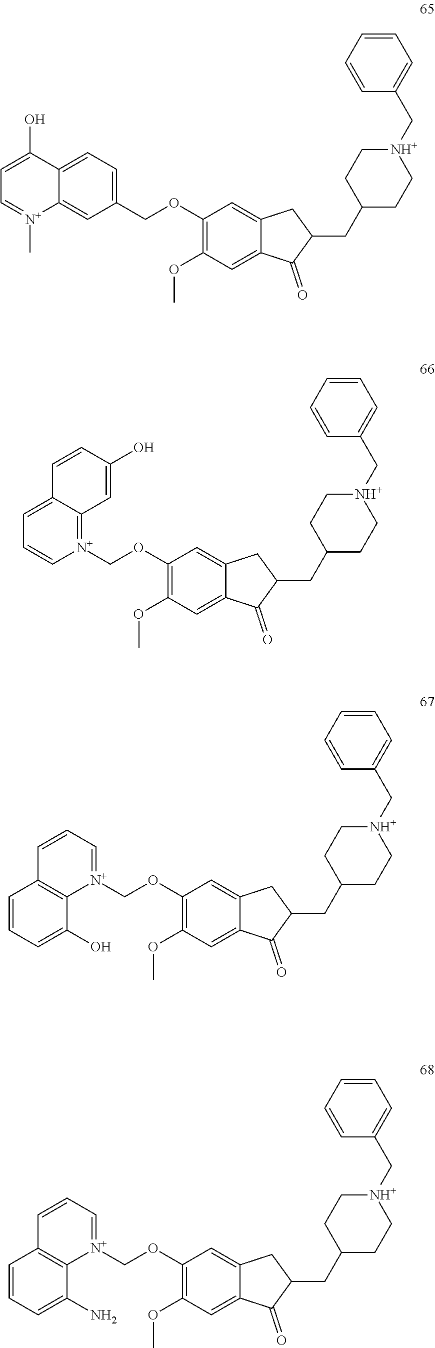 [SMILES: COc1cc2c(cc1OCc1ccc3c(O)cc[n+](C)c3c1)CC(CC1CC[NH+](Cc3ccccc3)CC1)C2=O ; COc1cc2c(cc1OC[n+]1cccc3ccc(O)cc31)CC(CC1CC[NH+](Cc3ccccc3)CC1)C2=O ; COc1cc2c(cc1OC[n+]1cccc3cccc(O)c31)CC(CC1CC[NH+](Cc3ccccc3)CC1)C2=O ; COc1cc2c(cc1OC[n+]1cccc3cccc(N)c31)CC(CC1CC[NH+](Cc3ccccc3)CC1)C2=O]

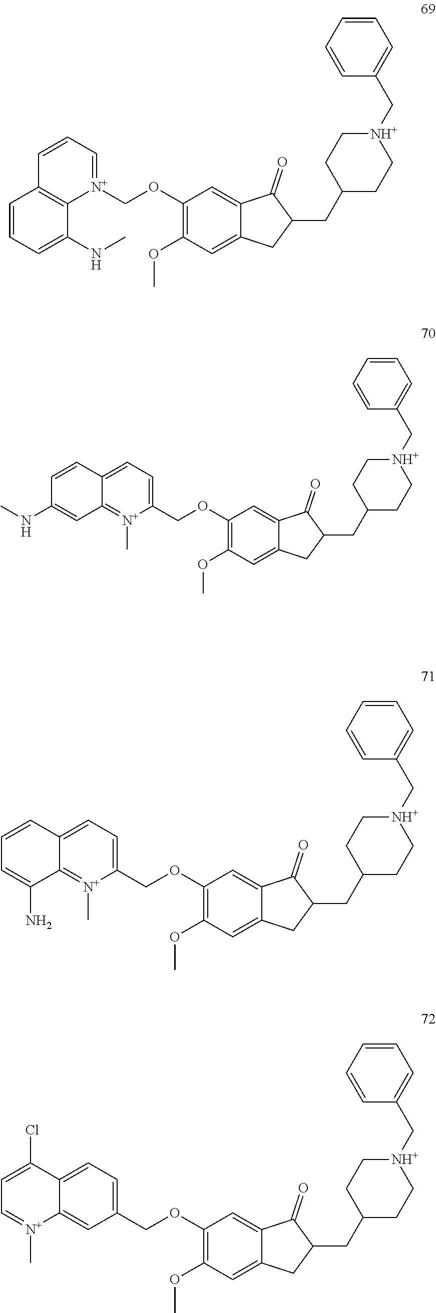 [SMILES: CNc1cccc2ccc[n+](COc3cc4c(cc3OC)CC(CC3CC[NH+](Cc5ccccc5)CC3)C4=O)c12 ; CNc1ccc2ccc(COc3cc4c(cc3OC)CC(CC3CC[NH+](Cc5ccccc5)CC3)C4=O)[n+](C)c2c1 ; COc1cc2c(cc1OCc1ccc3cccc(N)c3[n+]1C)C(=O)C(CC1CC[NH+](Cc3ccccc3)CC1)C2 ; COc1cc2c(cc1OCc1ccc3c(Cl)cc[n+](C)c3c1)C(=O)C(CC1CC[NH+](Cc3ccccc3)CC1)C2]

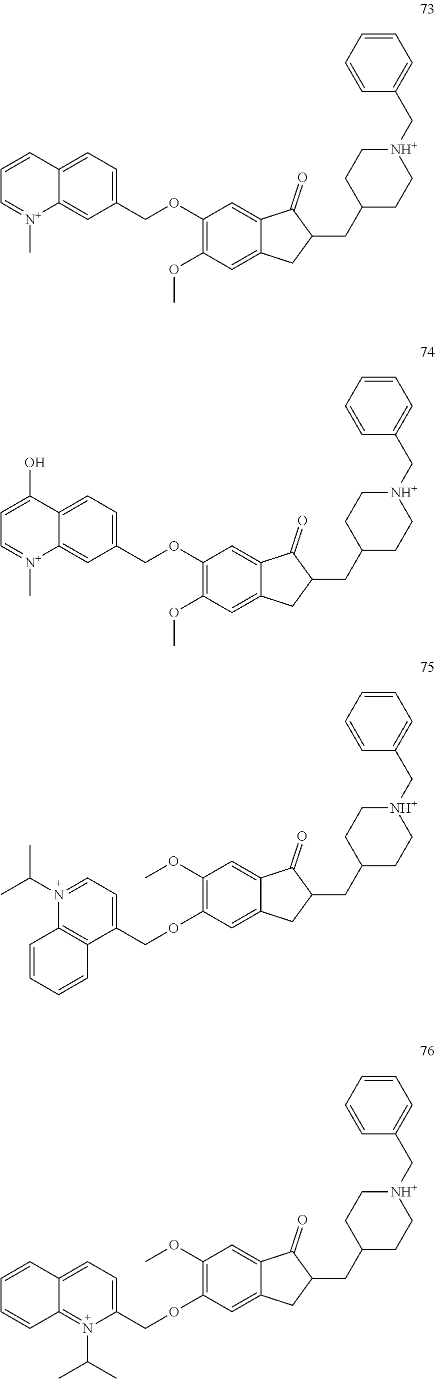

In a particular and preferred embodiment, the compound of formula (I) according to the invention is selected in the group consisting of:

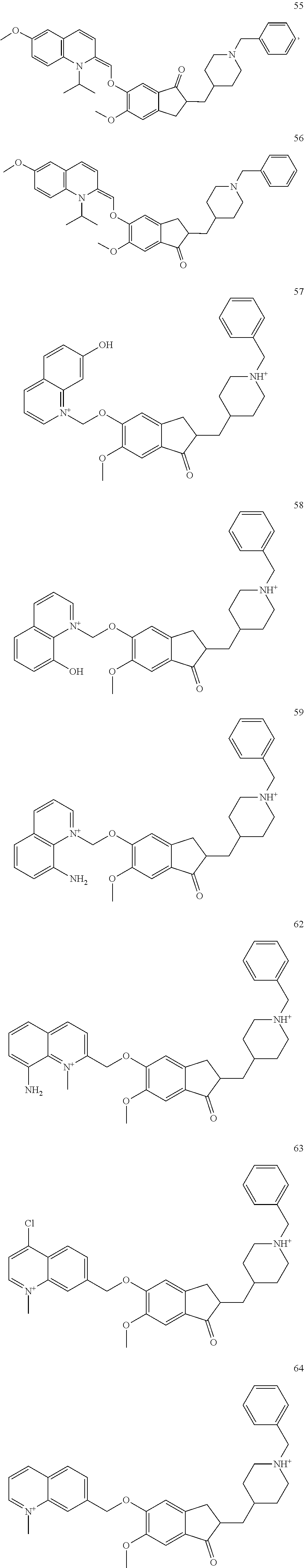

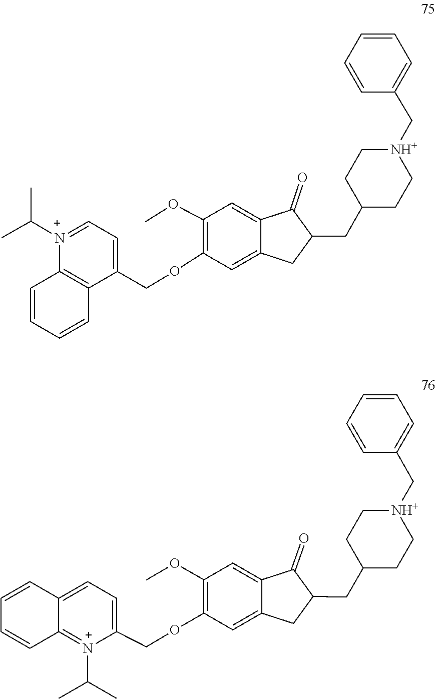

and the hydrochloride salts thereof.

In a more preferred embodiment, the compound of formula (I) according to the invention is:

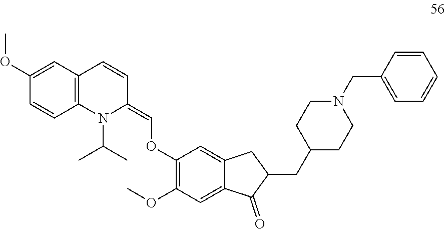 [SMILES: COc1ccc2c(c1)C=C/C(=C/Oc1cc3c(cc1OC)C(=O)C(CC1CCN(Cc4ccccc4)CC1)C3)N2C(C)C]

or the hydrochloride salt thereof.

### Method for Preparing a Compound of Formula (I)

The compound of formula (I) as described above, or a pharmaceutically acceptable salt and/or solvate thereof, may be obtained by conventional methods well-known in the art. In particular, compound of formula (I) as described above, or a pharmaceutically acceptable salt and/or solvate thereof, may be obtained by a method comprising the following steps:
(a) reacting a compound of formula (II)

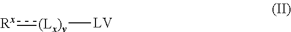

wherein [symbol]
is a single or a double bond,
R^{x} is an optionally substituted nitrogen-containing heterocyclyl group as defined above,
Lₓ is a divalent radical derived from a C₁-C₁₂ aliphatic chain as defined above,
v is 0 or 1 and
LV is a leaving group,
with a compound of formula (III) or (III′):

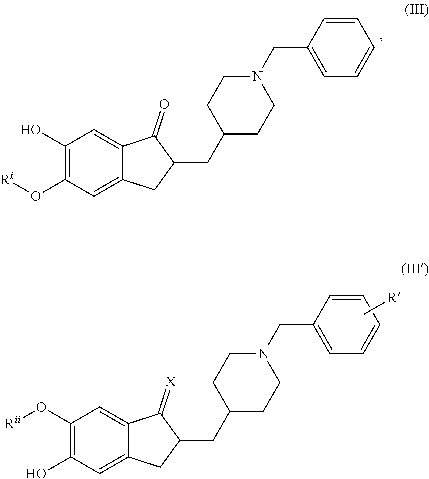

wherein
X is an oxygen atom or a group N—OH,
Rⁱ is R²-(L₂)ₙ as defined above, provided that R² is not an optionally substituted nitrogen-containing heterocyclyl group, in particular Rⁱ is a methyl group,
Rⁱⁱ is R¹-(L₁)ₚ as defined above, provided that R¹ is not an optionally substituted nitrogen-containing heterocyclyl group, in particular Rⁱⁱ is a methyl group,
and
R′ is as defined above,
(b) optionally, reacting compound of formula (III) or (III′) with a compound of formula (IV)

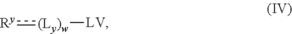

wherein
[symbol] is a single or a double bond,
   R^{u} is an optionally substituted nitrogen-containing heterocylyl as defined above,
   L_{y} is a divalent radical derived from a C₁-C₁₂ aliphatic chain as defined above,
   w is 0 or 1 and
   LV is a leaving group.

The compounds of formula (II), (III), (III′) and (IV) may be obtained according to methods well-known from the skilled person in the art. It is understood that the preferred embodiments recited for compound of formula (I) in the present disclosure are applicable to compounds (II), (III), (III′) and (IV).

It is understood that when compound of formula (II) reacts with compound of formula (III), Rx is typically as defined above for R¹, Lₓ is typically as defined above for L₁ and v is typically as defined above for p and when applicable, in compound of formula (IV), R^{y} is typically as defined above for R², L_{y} is typically as defined above for L₂ and n is typically as defined above for n.

It is also understood that when compound of formula (II) reacts with compound of formula (III′), Rx is typically as defined above for R², Lₓ is typically as defined above for L₂ and v is typically as defined above for n and when applicable, in compound of formula (IV), R^{y} is typically as defined above for R¹, L_{y} is typically as defined above for L₁ and n is typically as defined above for p.

LV is notably selected in the group consisting of halogen, preferably Cl, and sulfonate ester group such as mesylate or tosylate group.

In preferred embodiments, in compound of formula (III), Rⁱ is a methyl group and R′ is H. Synthesis of said preferred compounds of formula (III) is notably described in Wang, Wei; Guangdong Huagong, 2015, Vol. 42(17), p. 73-74 and EP296560 A2.

Optionally, additional steps of protection/deprotection and/or of functionalization well-known from the skilled person in the art may occur before or after the reaction between compounds of formula (II) and (III) or (III′) to afford compound of formula (I) with the suitable substituents as described above.

In particular, after step (a) and before step (b), the compound of formula (III) or (III′) may be deprotected on the position respectively bearing the substituent Rⁱ ou Rⁱⁱ, i.e. the group Rⁱ or Rⁱⁱ may be removed to afford an OH group on the corresponding position, so that said —OH may react with compound (IV) to afford a compound of formula (I) in which R¹ and R² are both a nitrogen-containing heterocyclyl group.

According to a preferred embodiment, only one from R¹ and R² is a nitrogen-containing heterocyclyl group group. According to this embodiment, step (b) is not performed and the group Rⁱ in the compound (III) corresponds to the group R²-(L₂)ₙ- in the final compound of formula (I) or the group Rⁱⁱ in the compound (III′) corresponds to the group R¹-(L₁)ₚ- in the final compound of formula (I).

According to a preferred embodiment, the compound of formula (III) or (III′) is obtained from donepezil by selectively deprotecting one the two methoxy groups bear by the indanone moiety, for example by using strong basic conditions. In this embodiment, Rⁱ or Rⁱⁱ respectively is a methyl group.

In the method of preparation of a compound of formula (I), an intermediate compound obtained at the end of a reacting step or the final compound obtained at the end of the reaction can be separated from the reaction medium by methods well known to the person skilled in the art, such as by extraction, evaporation of the solvent or by precipitation or crystallisation (followed by filtration).

Said compound can be also purified if necessary by methods well known in the art, such as by recrystallisation, by distillation, by chromatography on a column chromatography (for instance on silica gel) or by high performance liquid chromatography (HPLC).

### Pharmaceutical Composition

The present invention also relates to a pharmaceutical composition comprising at least one pharmaceutically acceptable excipient and at least one compound of formula (I) as described above or a pharmaceutically acceptable salt and/or solvate thereof.

According to some embodiments, the pharmaceutical composition of the present invention comprises from 0.1 mg to 5 mg, preferably from 0.1 mg to 2.5 mg of a compound of formula (I) or a pharmaceutically acceptable salt and/or solvate thereof.

The pharmaceutical compositions of the invention can be intended to oral or parenteral (including but not limited to subcutaneous, intramuscular, intravenous), intraperitoneal, ocular, intravitreal, topical, sublingual route of administration, preferably intraperitoneal, oral or intravenous administration. The active ingredient can be administered in unit forms for administration, mixed with conventional pharmaceutical carriers, to animals, preferably mammals including humans.

For oral administration, the pharmaceutical composition can be in a solid or liquid (solution or suspension) form.

A solid composition can be in the form of tablets, gelatin capsules, powders, granules and the like. In tablets, the active ingredient can be mixed with pharmaceutical vehicle(s) such as gelatin, starch, lactose, magnesium stearate, talc, gum arabic and the like before being compressed. The tablets may be further coated, notably with sucrose or with other suitable materials, or they may be treated in such a way that they have a prolonged or delayed activity. In powders or granules, the active ingredient can be mixed or granulated with dispersing agents, wetting agents or suspending agents and with flavor correctors or sweeteners. In gelatin capsules, the active ingredient can be introduced into soft or hard gelatin capsules in the form of a powder or granules such as mentioned previously or in the form of a liquid composition such as mentioned below.

A liquid composition can contain the active ingredient together with a sweetener, a taste enhancer or a suitable coloring agent in a solvent such as water. The liquid composition can also be obtained by suspending or dissolving a powder or granules, as mentioned above, in a liquid such as water, juice, milk, etc. It can be for example a syrup or an elixir.

For parenteral administration, the composition can be in the form of an aqueous suspension or solution which may contain suspending agents and/or wetting agents. The composition is advantageously sterile. It can be in the form of an isotonic solution (compared to blood).

### Therapeutic Uses

The compound of formula (I), the pharmaceutically acceptable salt and/or solvate thereof, or the pharmaceutical composition according to the invention act as acetylcholinesterase inhibitor, meaning that it is able to inhibit the hydrolysis of acetylcholine into choline and acetate thus allowing to increase the duration of action of acetylcholine in the central nervous system.

The present invention relates to a compound of formula (I) according to the invention or a pharmaceutically acceptable salt and/or solvate thereof or a pharmaceutical composition according to the invention for use as a drug, in particular in the prevention and/or the treatment of an acetylcholinesterase-associated disorder.

In other terms, the present invention relates to the use of a compound of formula (I) according to the invention or a pharmaceutically acceptable salt and/or solvate thereof, or a pharmaceutical composition according to the invention for the manufacture of a drug, notably intended in the prevention and/or the treatment of an acetylcholinesterase-associated disorder.

In other terms, the present invention relates to the use of a compound of formula (I) according to the invention or a pharmaceutically acceptable salt and/or solvate thereof or a pharmaceutical composition according to the invention for the prevention and/or the treatment of an acetylcholinesterase-associated disorder.

In other terms, the present invention relates to a method for the prevention and/or the treatment of an acetylcholinesterase-associated disorder comprising the administration to a person in need thereof of an effective dose of a compound of formula (I) according to the invention or a pharmaceutically acceptable salt and/or solvate thereof or a pharmaceutical composition according to the invention.

According to some embodiments, the compound of formula (I) or a pharmaceutically acceptable salt and/or solvate thereof for use in the prevention and/or the treatment of an acetylcholinesterase-associated disorder is administrated to a subject in need thereof at a daily dose ranging from 0.005 mg/kg to 0.5 mg/kg, preferably from 0.01 mg/kg to 0.1 mg/kg, more preferably from 0.01 mg/kg to 0.05 mg/kg, even more preferably from 0.01 mg/kg to 0.03 mg/kg.

An acetylcholine-associated disorder is preferably selected in the group consisting of compulsive disorders and related behaviors, including addiction, obsessive-compulsive disorders and eating disorders, and neurodegenerative disorders such as Parkinson's disease or Alzheimer's disease. The term “eating disorders” includes, in the context of the present invention anorexia, notably anorexia nervosa, bulimia, notably bulimia nervosa, and the compulsive dimension of obesity. By “compulsive dimension of obesity”, it is understood in the framework of the present invention that overweight obese patients often demonstrate compulsive eating behaviors (see Houben et al., Journal of Health Psychology, 2019 Volume 24, p. 1145-1152).

In particular, the compound of formula (I) according to the invention or a pharmaceutically acceptable salt and/or solvate thereof is useful for preventing or treating compulsive disorders and related behaviours, including addiction, obsessive-compulsive disorders and eating disorders, notably eating disorders such as anorexia nervosa and bulimia nervosa.

### DESCRIPTION OF THE FIGURES

FIG. 1: Experiment #1: Effect of daily-doses of 0.03 mg/kg of donquine (i.e. compound 56) and donepezil on VGLUT3^{T8I/T8I} mice and versus saline control in VGLUT3^{T8I/T8I} mice in an activity-based anorexia (ABA) model. A wild type (WT) group treated with saline was used as control. (A) Measure of food intake (% of baseline) during the 7 or 8 days of food restriction phase in WT mice (n=8, circles), VGLUT3^{T8I/T8I} mice daily-treated with 0.03 mg/kg of donquine (n=7, triangles) and VGLUT3^{T8I/T8I} mice daily-treated with saline (n=8, squares). (B) Bodyweight was monitored daily and mice having less than 75% body weight from baseline were considered anorexic. During food restriction phase of the ABA test: percentage of mice having a bodyweight equal or greater than 75% of their baseline bodyweight in WT mice (n=8, solid line), in VGLUT3^{T8I/T8I} mice daily-treated with 0.03 mg/kg of donquine (n=7, dotted line) and in VGLUT3^{T8I/T8I} mice treated with saline (n=8, dashes). Experiment #2: Effect of daily-doses of 0.01 mg/kg of donquine (i.e. compound 56) versus donepezil (0.03 mg/kg) and saline control in VGLUT3^{T8I/T8I} mice in an activity-based anorexia (ABA) model. (C) Measure of food intake (% of baseline) during the 7 or 8 days of food restriction phase in VGLUT3^{T8I/T8I} mice daily-treated with 0.01 mg/kg of donquine (n=7, triangles), VGLUT3^{T8I/T8I} mice daily-treated with 0.03 mg/kg of donepezil (n=8, squares) and VGLUT3^{T8I/T8I} mice treated with saline (n=8, circles). (D) During food restriction phase of the ABA test: percentage of mice having a weight equal or greater than 75% of their baseline bodyweight in VGLUT3^{T8I/T8I} mice treated with saline (n=8, solid line), in VGLUT3^{T8I/T8I} mice daily-treated with 0.01 mg/kg of donquine (n=7, dotted line) and in VGLUT3^{T8I/T8I} mice daily-treated with 0.03 mg/kg of donepezil (n=8, dashes).

### EXAMPLES

### 1) Synthesis

### 1.1) Material

Air and moisture sensitive manipulations were performed either under nitrogen or in vacuo using standard Schlenk techniques. Anhydrous solvents (Et₂O, EtOH, DCM, MeOH CH₃CN, THF, toluene, and hexane) were purchased from Sigma-Aldrich (Merck KGaA, Darmstadt, Germany). All other chemicals were purchased from Alfa Aesar (Karlsruhe, Germany), Sigma Aldrich and TCI Europe (Boerenveldseweg, Belgium) and used without further purification unless otherwise stated. Analytical thin layer chromatography (TLC) was performed with Merck SIL G/UV254 plates. Compounds were visualized by exposure to UV light or by dipping the plates in solutions of phosphomolybdic acid, ninhydrin or potassium permanganate followed by heating. Flash column chromatography was performed with silica gel 60 (Sigma-Aldrich, St Louis, USA). NMR spectra were recorded on Brucker Avance Ill nanobay 300 MHz or 400 MHz spectrometers. ¹H- and ¹³C-NMR chemical shifts (6) are quoted in parts per million (ppm) relative to the TMS scale. Coupling constants J are quoted in Hz. The following abbreviations are used for the proton spectra multiplicities: s: singlet, d: doublet, t: triplet, q: quartet, qt: quintuplet, sp: septuplet, m: multiplet, br.: broad, dd: double doublet, dt: double triplet. Coupling constants (J) are reported in Hertz (Hz). Mass spectrometry was carried out at the University of Paris Descartes mass spectrometry service, either in perfusion mode or using a Shimadzu Nexera X2 HPLC system (Kyoto, Japan) and a High Resolution Orbitrap Exactive Mass Spectrometer (Thermo Fisher, San Jose, USA). HPLC purification was performed with a Gilson analytical instrument (Villiers-le-Bel, France).

### 1.2) Synthesis and Characterization

### General Procedure A

### Step 1: Synthesis of iodo-1-alkyl quinolinium derivative

Optionally substituted quinoline (5 g, 31.40 mmol) was refluxed with appropriate iodoalkyl (15.6 mL, 157 mmol) for 48 h. The reaction mixture was cooled to room temperature and Et₂O was added. The mixture was triturated then the residue was washed with Et₂O to yield the corresponding 1-alkyl-quinolinium iodide as solid. The compound was used without further purification. The optionally substituted 1-alkyl-quinolinium iodide was then added over 5 min to a solution of MeMgBr (3 M in DCM, 19.43 mL, 58.3 mmol) at 0° C. The reaction mixture was stirred at this temperature for 1 h then 2 h at room temperature. Water was added slowly, followed by a solution of concentrated HCl until the formation of two layers, then the addition of ammonium chloride, and the solution was made alkaline with ammonia. The organic layer was washed with water, dried over MgSO₄ and concentrated in vacuo to yield the appropriate dihydroquinoline, which was directly used for the next step without purification. The dihydroquinoline was refluxed in EtOH (40 mL) with iodine (9.6 g) for 15 min and cooled to room temperature. The resulting residue was filtered and washed with EtOH and Et₂O to obtain the corresponding 1-alkyl-methylquinolin-1-ium iodide. The optionally substituted 1-alkyl-methylquinolin-1-ium iodide 100 mg (0.29 mmol, 1 eq) was dissolved in 15 mL of dichloromethane (DCM), colour brown-yellow. After dissolution, approximately 90 μL (0.64 mmol, 2.2 eq) of triethylamine (TEA) was added. The red-brown solution darkened to brown-black colour. After five minutes, iodine (0.29 mmol, 1 eq) were added. After 3 hours iodine was readded (0.29 mmol, 1 eq). The reaction was stirred for 60 hours at room temperature. The mixture was purified as follows: Solvent was removed in vacuo (black-brown oily liquid). 5 mL cyclohexane was added, which turned pink. Cyclohexane was removed after 1 hour and the rest of it was removed in vacuo. The reaction mixture was diluted with DCM (30 mL) and washed with sat. NaCl (2×20 mL). Solvent was removed in vacuo. TLC and ninhydrin test—showed triethylamonium in water. NMR spectrum still showed an excess of TEA. The reaction mixture was diluted with DCM (3 mL) and filtrated through 1 cm silica gel DCM/MeOH (1:0→95:5). Usually there is no need of purification the crude product is used for the addition on the abacavir or formed in situ.

### Specific Example: Synthesis of Intermediate H2-QUIN

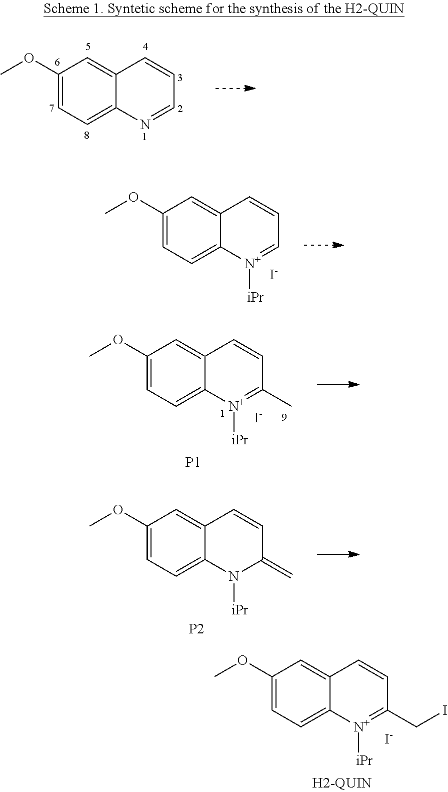

### 6-methoxy-1-isopropylquinolin-1-ium iodide

6-methoxyquinoline (5 g, 31.40 mmol) was refluxed with iodopropane (15.6 mL, 157 mmol) for 48 h. The reaction mixture was cooled to room temperature and Et₂O was added. The mixture was triturated then the residue was washed with Et₂O to yield the title compound (9.4 g, 91%) as a yellow solid; ¹H-NMR (400 MHz, DMSO-d6) δ 9.44 (dd, 1H, J=1.5, 6.0 Hz, ArH), 9.11 (d, 1H, J=8.4 Hz, ArH), 8.67 (d, 1H, J=9.9 Hz, ArH), 8.14 (dd, 1H, J=6.0, 8.3 Hz, ArH), 7.94 (d, 1H, J=3.0 Hz, ArH), 7.90 (dd, 1H, J=7.3, 9.0 Hz, ArH), 5.86 (h, 1H, J=6.5 Hz, CHCH₃), 4 (s, 3H, OCH₃), 1.70 (d, 6H, J=6.5 Hz, CHCH₃).

### 1-isopropyl-6-methoxy-2-methylquinolin-1-ium iodide P1

1-isopropyl-6-methoxyquinolin-1-ium iodide (10 g, 29.15 mmol) was added over 5 min to a solution of MeMgBr (3 M in DCM, 19.43 mL, 58.3 mmol) at 0° C. The reaction mixture was stirred at this temperature for 1 h then 2 h at room temperature. Water was added slowly, followed by a solution of concentrated HCl until the formation of two layers, then the addition of ammonium chloride, and the solution was made alkaline with ammonia. The organic layer was washed with water, dried over MgSO₄ and concentrated in vacuo to yield the dihydroquinoline (6.02 g), which was directly used for the next step without purification. The dihydroquinoline was refluxed in EtOH (40 mL) with iodine (9.6 g) for 15 min, and cooled to room temperature. The resulting residue was filtered and washed with EtOH and Et₂O to obtain the 1-isopropyl-6-methoxy-2-methylquinolin-1-ium iodide (9.2 g) in 88% yield; ¹H-NMR (400 MHz, DMSO-d6) δ 8.93 (d, 1H, J=8.6 Hz, ArH), 8.65 (d, 1H, J=9.9 Hz, ArH), 8.04 (d, 1H, J=8.5 Hz, ArH), 7.85 (d, 1H, J=3.1 Hz, ArH), 7.74 (d, 1H, J=8.5 Hz, ArH), 5.65 (sp, 1H, J=6.5 Hz, CHCH₃), 3.99 (s, 3H, CH₃), 1.85 (d, 6H, J=6.5 Hz, CHCH₃).

### Synthesis of 2-(iodomethyl)-1-isopropyl-6-methoxyquinolin-1-ium iodide (H2-QUIN)

The 1-isopropyl-6-methoxy-2-methylquinolin-1-ium iodide 100 mg (0.29 mmol, 1 eq) was dissolved in 15 mL of dichloromethane (DCM), colour brown-yellow. After dissolution, approximately 90 μL (0.64 mmol, 2.2 eq) of triethylamine (TEA) was added. The red-brown solution darkened to brown-black colour. After five minutes 74 mg (0.29 mmol, 1 eq) of iodine were added. After 3 hours 74 mg (0.29 mmol, 1 eq) of iodine were added. The reaction was stirred for 60 hours at room temperature. The mixture was purified as follows: Solvent was removed in vacuo (black-brown oily liquid). 5 mL cyclohexane was added, which turned pink. Cyclohexane was removed after 1 hour and the rest of it was removed in vacuo. The reaction mixture was diluted with DCM (30 mL) and washed with sat. NaCl (2×20 mL). Solvent was removed in vacuo. TLC and ninhydrin test—showed triethylamonium in water. NMR spectrum still showed an excess of TEA. The reaction mixture was diluted with DCM (3 mL) and filtrated through 1 cm silica gel DCM/MeOH (1:0→95:5). Usually there is no need of purification the crude product is used for the addition on the abacavir or formed in situ.

### Step 2: Coupling with Donepezil

Donepezil (1 g, 2.6 mmol) was demethylated on position 5 by reacting with NaCN (1.25 g, 26 mmol) in DMSO (10 mL). The reaction was stirred at 100° C. for two days, cooled, diluted with water and extracted with dichloromethane. The aqueous solution was acidified with concentrated HCl, and extracted with dichloromethane. The organic layer was dried over MgS04 and evaporated to give the corresponding intermediate (500 mg).

In a flask under argon equipped with a magnetic stirring bar, the intermediate (61 mg, 0.164 5 mmol), silver acetate (AgOAc) (2.13 mg, 12.8 μmol) and 1,2-bis(diphenylphosphino)ethane (dppe) (5.1 mg, 12.8 μmol) are placed. 0.64 mL of DMF are added afterwards. The mixture is placed under stirring at room temperature for 10 minutes and then at −10° C. Then the iodo-1-alkyl quinolinium derivative of step 1 (0.1 g, 0.213 mmol) and LiHMDS (2.8 mg, 17 μmol) are added. The reaction medium is left under these conditions for 8 hours until the reagents are consumed. Finally according the thin-layer chromatography, we did a fraction separation by column chromatography (Cyclohexane/EtOAc 7:3). Compounds were obtained with 3-10% yield.

### Specific Example: Synthesis of Compound 56

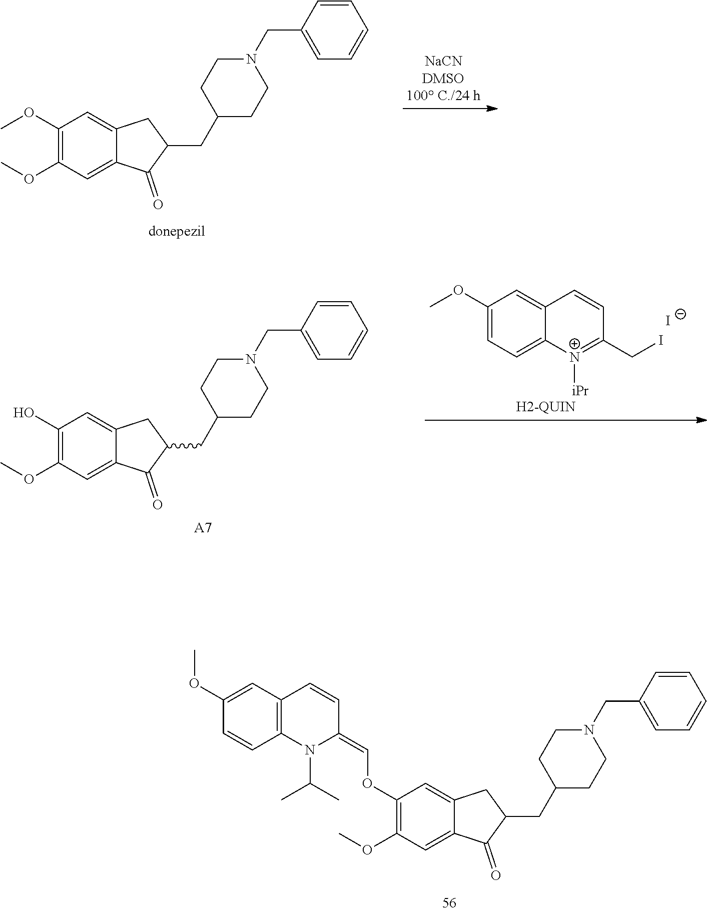 [SMILES: COc1cc2c(cc1OC)C(=O)C(CC1CCN(Cc3ccccc3)CC1)C2 ; COc1cc2c(cc1O)CC(CC1CCN(Cc3ccccc3)CC1)C2=O ; COc1ccc2c(ccc(CI)[n+]2C(C)C)c1 ; [I-] ; "A7" ; COc1ccc2c(c1)C=C/C(=C/Oc1cc3c(cc1OC)C(=O)C(CC1CCN(Cc4ccccc4)CC1)C3)N2C(C)C]

### Intermediate A7:

¹H-NMR (400 MHz, CDCl₃) δ 7.30-7.20 (m, 3H, aromatic), 7.19-7.15 (m, 2H, aromatic), 7.10 (s, 1H, aromatic), 6.85 (s, 1H, aromatic), 3.85 (s, 3H, OCH3), 3.43 (s, 2H, NCH2Ph), 3.15 (dd, 1H), 2.90-2.80 (m, 2H), 2.66 (m, 2H), 1.93-1.83 (m, 3H), 1.69-1.55 (m, 2H), 1.40 (m, 1H), 1.35-1.15 (m, 3H).

### Compound 56: (Yield=10%)

¹H-NMR (400 MHz, CDCl₃) δ δ 9.34 (d, 1H, NH), 8.80 (d, 1H, aromatic), 8.47 (d, 1H, aromatic), 8.41 (d, 1H, aromatic), 8.56 (s, 1H, aromatic), 8.26 (s, 1H, aromatic), 7.94 (d, 1H, aromatic), 7.50-7.45 (m, 2H, aromatic), 7.40-7.35 (m, 2H, aromatic), 7.15 (s, 1H, aromatic), 6.92 (s, 1H, aromatic), 4.05 (s, 3H, OCH3); 3.96 (s, 3H, OCH3), 3.13 (s, 2H, NCH2Ph), 3.05 (s, 1H); 3.01 (m, 1H), 2.90-2.80 (m, 2H), 2.67 (m, 2H), 2.05-1.80 (m, 3H), 1.69-1.55 (m, 2H), 1.40 (m, 1H), 1.35-1.20 (m, 3H).

### Characterization of Compounds of the Invention

### Compound 62: (Yield=3.5%)

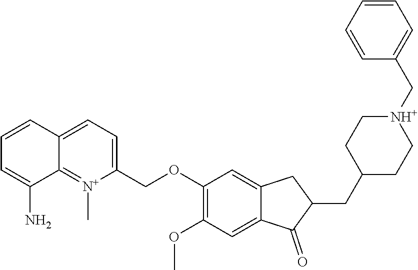

¹H-NMR (400 MHz, CDCl₃) δ 9.77 (d, 1H, NH), 8.91 (d, 1H, aromatic), 8.23-8.09 (m, 1H, aromatic), 7.86 (d, 2H, aromatic), 7.70 (m, 2H, aromatic), 7.40-7.35 (m, 2H, aromatic), 7.17 (s, 1H, aromatic), 6.89 (s, 2H, aromatic), 5.92 (s, 2H, CH2); 4.68 (s, 3H, OCH3), 3.13 (s, 2H, NCH2Ph), 3.05 (s, 1H); 3.01 (m, 1H), 2.90-2.80 (m, 2H), 2.67 (m, 2H), 2.05-1.80 (m, 3H), 1.69-1.55 (m, 2H), 1.45 (m, 3H).

### Compound 64: (Yield=4.20%)

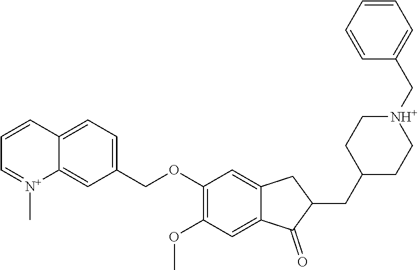

¹H-NMR (400 MHz, CDCl₃) δ 8.95 (d, 1H), 8.41-8.16 (m, 3H, aromatic), 8.08 (m, 1H) 7.85 (m, 1H), 7.75 (m, 1H), 7.62 (m, 1H); −7.30 (m, 5H), 7.13 (d, 1H), δ 0.94 (s, 1H); 5.00 (s, 2H); 3.86 (s, 3H); 3.75 (s, 3H); 3.16 (s, 2H); 3.22 (3H); 2.84-2.51 (m, 4H); 2.05-1.85 (m, 2H); 1.59-1.25 (m, 5H).

### Compound 63: (Yield=3.26%)

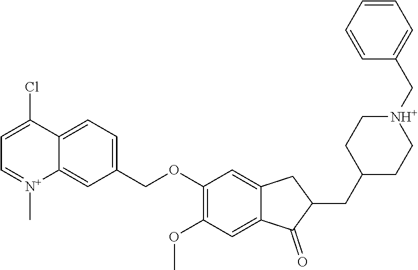

¹H-NMR (400 MHz, CDCl₃) δ 8.30 (d, 2H), 8.00 (m, 2H), 7.82 (s, 1H), 7.48 (m, 2H), 7.35-7.15 (m, 6H), 6.93 (s, 1H); 6.31 (s, 2H); 3.94 (m, 6H); 3.53 (s, 2H); 3.35 (1H); 2.92-2.85 (m, 2H); 2.15-1.90 (m, 4H); 1.57 (m, 2H), 1.37-1.20 (m, 5H).

### Compound 75 (Yield=9.15%)

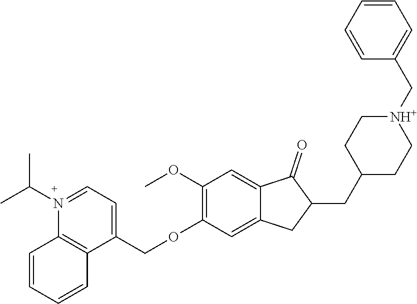

¹H-NMR (400 MHz, CDCl₃) δ 7.70 (d, 1H), 7.62 (d, 2H, aromatic), 7.53-7.32 (m, 2H) 7.85 (m, 2H), 7.62 (m, 2H), 7.52-7.30 (m, 3H), 7.21 (m, 1H), 6.91 (m, 1H); 4.26 (m, 1H); 3.98 (s, 3H); 3.87 (s, 2H); 3.70-3.27 (3H); 2.60-2.34 (m, 4H); 2.36-2.21 (m, 2H); 1.61-1.28 (m, 5H); 0.98-0.82 (d, 6H).

### Compound 76 (Yield=6.48%)

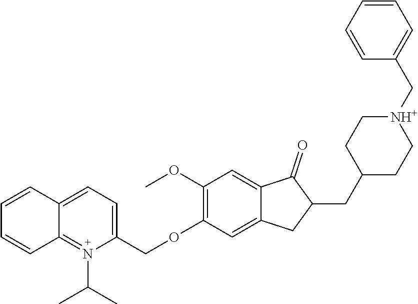

¹H-NMR (400 MHz, CDCl₃) δ 8.92 (s, 2H), 8.13 (m, 3H), 7.85 (m, 1H), 7.71 (m, 1H), 7.52-7.30 (m, 5H), 7.13 (d, 1H), δ 0.82 (s, 1H); 5.23 (s, 3H); 3.92 (s, 3H); 3.48 (m, 3H); 3.12 (1H); 2.86 (m, 1H); 2.63-2.30 (m, 4H); 2.25 (m, 2H), 1.37-1.20 (m, 11H).

### General Procedure B

Donepezil (1 g, 2.6 mmol) was demethylated on position 5 by reacting with NaCN (1.25 g, 26 mmol) in DMSO (10 mL). The reaction was stirred at 100° C. for two days, cooled, diluted with water and extracted with dichloromethane. The aqueous solution was acidified with concentrated HCl, and extracted with dichloromethane. The organic layer was dried over MgS04 and evaporated to give the corresponding intermediate (500 mg).

In a flask under argon equipped with a magnetic stirring bar, the intermediate (61 mg, 0.164 mmol), was stirred for 3 h at room temperature with K2CO3 (25 mg, 0.25 mmol, 1.5 equiv), iodo methyl bromide (4 equiv), and DMF (2 mL). The mixture was then extracted with dichloromethane and after evaporation the crude compound was used without further purification with the quinoline derivative. The quinoline derivative (3.14 mmol, 2 eq) was refluxed with crude iodo-donepezil derivative (15.7 mmol, 5 eq) in anhydrous THF for 48 h. The reaction mixture was cooled to room temperature and Et₂O was added. The mixture was triturated then the residue was washed with Et₂O to yield the title compound as a brown solid with 2.45-10.3% yield.

### Compound 59: (Yield=5.43%)

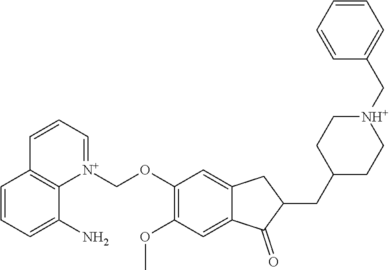

¹H-NMR (400 MHz, CDCl₃): δ 7.65 (m, 3H), 7.49 (m, 5H), 7.21-7.09 (m, 2H), 6.92 (s, 1H), 5.38 (s, 2H), 5.31 (s, 1H), 4.19 (s, 3H), 3.49 (s, 2H), 3.22 (m, 1H), 2.90-2.40 (m, 4H), 2.60-2.45 (m, 4H), 2.05-1.80 (m, 2H), 1.57-1.35 (m, 2H), 1.30-1.10 (m, 3H).

### Compound 58: (Yield=2.45%)

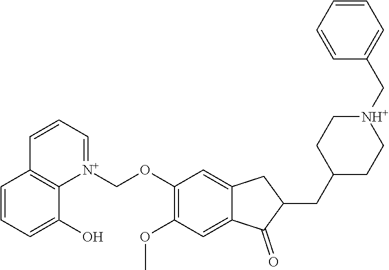

¹H-NMR (400 MHz, CDCl₃) δ 8.94 (d, 1H), 8.78 (d, 1H, aromatic), 8.18 (m, 2H, aromatic), 7.75 (d, 1H, aromatic), 7.65-7.35 (m, 6H, aromatic), 7.19 (s, 1H, aromatic), 7.13 (s, 1H, aromatic), 5.65 (s, 2H, CH2); 3.91 (s, 3H); 3.71 (s, 2H), 3.55 (m, 1H); 2.95 (s, 2H); 2.60-2.22 (m, 6H), 1.80-1.54 (m, 5H).

### Compound 57: (Yield=10.3%)

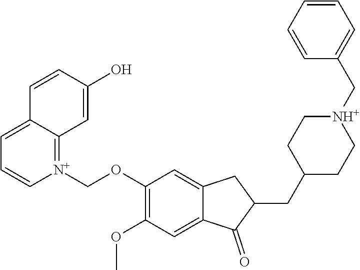

¹H-NMR (400 MHz, CDCl₃) δ 8.95 (d, 1H), 8.19 (d, 2H, aromatic), 8.08 (m, 2H) 7.85 (m, 2H), 7.62 (m, 2H), 7.52-7.30 (m, 5H), 7.13 (d, 1H), δ 0.98 (s, 1H); 5.44 (s, 2H); 4.10 (s, 2H); 3.92 (s, 3H); 3.48 (m, 1H); 3.24 (2H); 2.6-2.34 (m, 4H); 2.01-1.82 (m, 2H); 1.61-1.28 (m, 5H).

2) Efficacy of Donquine (i.e. Compound 56) in Ach-Deprived Mutant Mice in the ABA Model

Self-starvation behaviors are modeled in the activity-based anorexia (ABA) test (Klenotich, S. J., Dulawa, 2012 Methods Mol Biol 829, 377-93). In this test, animals are housed in the presence of a running wheel. Access to food is progressively restricted over a period of 8 days. For these animal experiments, an hypocholinergic mouse model was used. The type 3 atypical vesicular transporter (VGLUT3) is expressed by all cholinergic striatal interneurons (El Mestikawy et al. 2011, Nat Rev Neurosci 12(4), 204-16).

In 2015, the El Mestikawy team discovered a p.T8I variant of VGLUT3 expressed in patients suffering from substance use disorder and from eating disorders (Sakae et al. Mol Psychiatry, 2015, 20(11), 1448-59). Mice expressing this variant (VGLUT3^{T8I/T8I} mice) have a reduced cholinergic tone in the striatum and are more vulnerable to self-starvation in the ABA model. In other terms, VGLUT3^{T8I/T8I} mice present the same main symptomology than anorexic patients and are considered to be a model of choice for anorexia.

### Activity-Based Anorexia (ABA) Model

The ABA model was performed as described by Klenotich and Dulawa (Klenotich, S. J., Dulawa, 2012 Methods Mol Biol 829, 377-93) and as previously reported (Favier, M., et al., J Clin Invest 130, 6616-6630 (2020)). For habituation, all mice were individually housed in cages with running wheels for 7 days with unrestricted access to food, water and running wheel. After the adaptation period, all mice were maintained in the same running wheel cages for 8 additional days. Access to food was progressively restricted, from 8 h (day 1) to 2 h (day 8) per day. Body weight and food intake were measured daily before and after food access, respectively. Mice losing more than 25% of their initial (baseline) body weight are considered “anorexic”. Days until mice reached 75% or less of baseline BW provided a measure of survival. For pharmacological treatment experiment, mice received daily intraperitoneal injection of donquine (i.e. compound 56, at 0.03 or 0.01 mg/kg, diluted in NaCl 0.9%)), donepezil (compound of reference, at 0.03 mg·kg⁻¹, diluted in NaCl 0.9%) or control saline (NaCl) solution at 0.9 wt %. Mice were treated daily 30 min before the start of food access, during both baseline and food restriction phases.

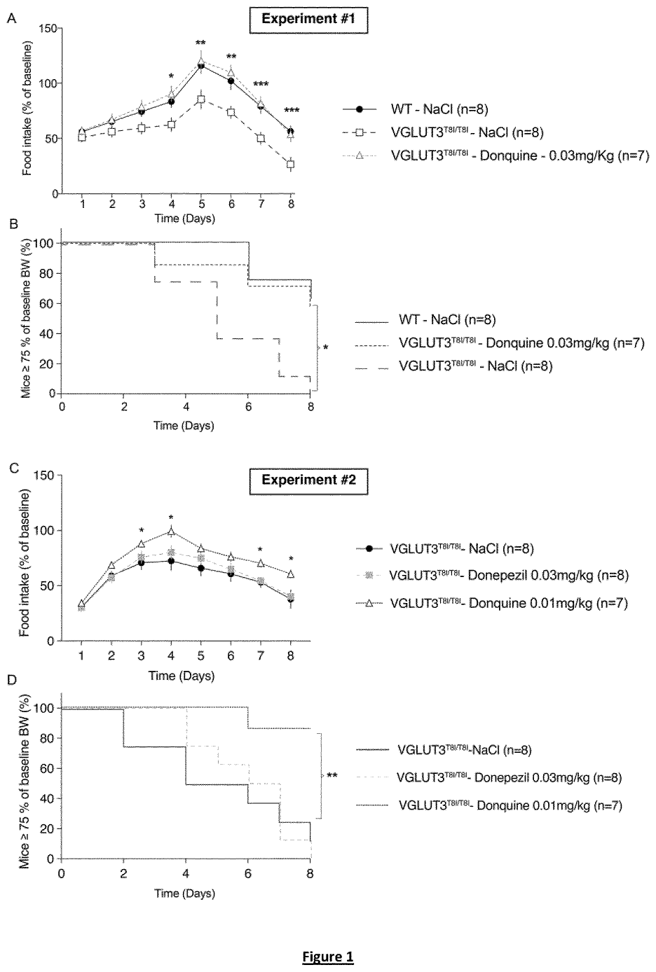

FIG. 1(A) illustrates that the administration of donquine (i.e. compound 56) at a dose of 0.03 mg/kg each day for 7 days to the VGLUT3^{T8I/T8I} mice leads to similar level of food intake than in wild type mice treated with saline. By contrast, VGLUT3^{T8I/T8I} mice treated with saline tend to eat less and present a self-starvation/anorexic-like behavior.

FIG. 1(B) shows that all (100%) of VGLUT3^{T8I/T8I} mice treated with saline have less than 75% of their baseline body weight after 8 days in the ABA test. By contrast, the administration of daily-doses at 0.03 mg/kg of donquine prevents VGLUT3^{T8I/T8I} mice from losing weight as 60% of them still weigh at least 75% of their baseline body weight after 8 days, which is similar to the control wild-type group treated with saline. Similarly, FIG. 1C shows that daily-doses at 0.01 mg/kg of donquine also rescued mice from self-starvation as about 90% of mice weigh at least 75% of their baseline body weight after 8 days. Altogether, this data exemplifies that donquine is a suitable and effective treatment for anorexic behaviors.

In comparison, same experiment has been performed with a known acetylcholinesterase inhibitor, the donepezil (see FIGS. 1(C) and 1(D)). FIG. 1(C) shows that donquine, at a lower dose (0.01 mg/kg), is more efficient than donepezil at 0.03 mg/kg in reducing the food restriction in VGLUT3^{T8I/T8I} mice. FIG. 1(D) shows that a daily dose of 0.03 mg/kg of donepezil is less efficient to reduce the number of VGLUT3^{T8I/T8I} mice losing weight until 75% of their baseline bodyweight or less than a daily dose of 0.01 mg/kg of donquine.

### 3) Inhibition of Acetylcholinesterase (AChE)

### 3.1) Procedure

A solution of each tested compound is prepared by dissolving the compound in DMSO at 1 mM stock solution and diluting with water to the following concentration 100 nM, 10 nM and 1 nM.

The procedure of MAK119 kit assay of Sigma Aldrich (colorimetric test) has been followed as described:

The Working Reagent (MAK119C) of the kit is prepared fresh and used within 30 minutes. A solution A is prepared by dissolving the Working Reagent (192 mg) in 19.2 ml of Assay Buffer (MAK119A) of the kit.

A solution B made with 200 mL of water and 200 mL of Calibrator (MAK119B of the kit) is distributed into separate wells of the 96-well plate. 10 mL of the solution of the tested compound is added into wells and then 190 mL of the solution A is added to all sample wells. Negative control wells are prepared by adding solutions A and B without adding solution of the tested compound and positive control wells are prepared by adding solutions A, B and donepezil 1 μM.

The plate was incubated at room temperature for 2 min and the initial absorbance at 412 nm (A₄₁₂)ᵢₙᵢ was measured. After 10 min of incubation at room temperature the final measurement (A₄₁₂)_{final}.

The following calculation is then achieved: (A₄₁₂)_{blank} Inhibition was calculated by comparing the absorbance of the samples containing the tested compounds at 412 nm (A₄₁₂) and of the absorbance of the negative and positive controls after 12 min of incubation.

### 3.2) Results

| Tested | % Inhibition | % Inhibition | % Inhibition |
|---|---|---|---|
| compound | (100 nM) | (10 nM) | (1 nM) |
| 56 | 100 | 75 | 50 |
| 62 | 100 | 100 | |
| 59 | 100 | 100 | |
| 57 | 100 | 100 | |
| 76 | 100 | 100 | |
| 63 | 75 | 75 | 75 |
| 64 | 100 | 100 | 25 |
| 75 | 100 | 100 | |
| Donepezil | 100 | 75 | 25 |

These results show that the compounds of the invention are at least as efficient, and for some of them more efficient, than donepezil in inhibiting acetylcholinesterase.

1. A compound of formula (I): 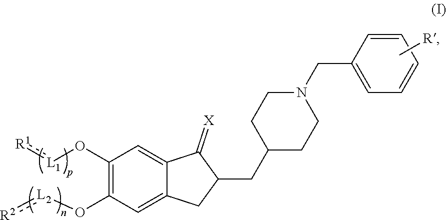
or a pharmaceutically acceptable salt and/or solvate thereof,
in which [symbol]
represents a single or a double bond, and
X is an oxygen atom or a group N—OH,
R¹ and R² are each independently H, an optionally substituted nitrogen-containing heterocyclyl group, an optionally substituted C₁-C₆ aliphatic chain or an optionally substituted aryl, wherein up to 4 methylene units of the C₁-C₆ aliphatic chain are optionally replaced by 0, C(O), NH or N—C₁-C₆alkyl, provided that at least one of R¹ and R² is an optionally substituted nitrogen-containing heterocyclyl group,
L₁ and L₂ are each independently a divalent radical derived from a C₁-C₁₂ aliphatic chain, wherein one or more methylene unit(s) are optionally replaced by arylene, —O—, —S—, —C(═O)—, —SO₂— or —N(C₁-C₆ alkyl)-, wherein the C₁-C₁₂ aliphatic chain is optionally substituted,
p and n are each independently 0 or 1, provided that p is 1 when R¹ is an optionally substituted nitrogen-containing heterocyclyl group and n is 1 when R² is an optionally substituted nitrogen-containing heterocyclyl group, and
R′ is H, halogen, an optionally substituted C₁-C₆ aliphatic chain, an optionally substituted aryl, an optionally substituted heteroaryl or an optionally substituted C₁-C₆ alkyl-aryl, wherein up to 4 methylene units of the C₁-C₆ aliphatic chain are optionally replaced by O, C(O), NH or N—C₁-C₆alkyl.

2. The compound of claim 1, wherein the optionally substituted nitrogen-containing heterocyclyl group is selected from the group consisting of pyrrolyl, pyridyl, thiazinyl, thiazolyl, isothiazolyl, imidazolyl, pyrazolyl, triazolyl, tetrazolyl, pyridazinyl, purinyl, pyrimidinyl, pyrazinyl, triazinyl, quinolinyl, isoquinolinyl, isoindolyl, indolyl, piperidinyl, piperizinyl, pyrrolidinyl, pyrazolidinyl, imidazolidinyl, thiazolidinyl, isothiazolidinyl, dihydroquinolinyl, dihydroisoquinolinyl, tetrahydroquinolinyl, tetrahydroisoquinolinyl, indolinyl and isoindolinyl.

3. The compound of claim 1, R¹ and/or R² is a group corresponding to one of the following formula: 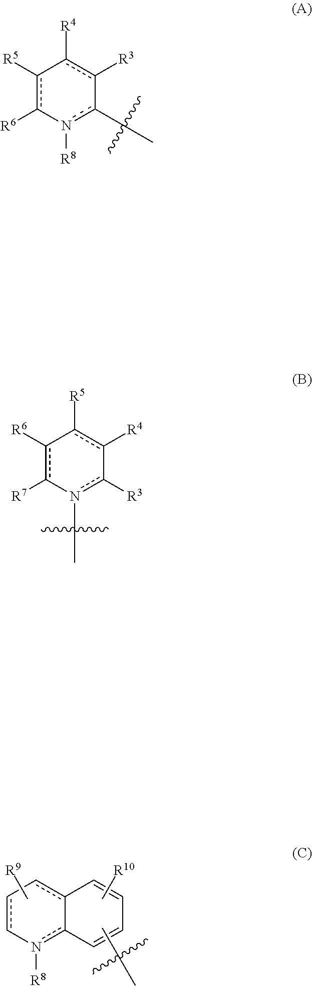
in which
R³ to R⁸ are each independently selected in the group consisting of H, halogen, OH, NH₂, NH—C₁-C₆ alkyl, a C₁-C₆ aliphatic chain, aryl, heteroaryl and C₁-C₆ alkyl-aryl, wherein up to 4 methylene units of the C₁-C₆ aliphatic chain are optionally replaced by O, C(O), NH or N—C₁-C₆alkyl, the C₁-C₆ aliphatic chain, aryl, heteroaryl or alkyl-aryl being optionally substituted, or
one or more among the couples R³-R⁴, R⁴-R⁵, R⁵-R⁶ and R⁶-R⁷ form together with the carbon atoms to which they are bonded a 5 or 6 membered aromatic or non aromatic optionally substituted ring,
R⁹ and R¹⁰ are each independently one or more substituents selected in the group consisting of H, a C₁-C₆ aliphatic chain, aryl, heteroaryl and C₁-C₆ alkyl-aryl, wherein up to 4 methylene units of the C₁-C₆ aliphatic chain are optionally replaced by O, C(O), NH or N—C₁-C₆alkyl, the C₁-C₆ aliphatic chain, aryl, heteroaryl or alkyl-aryl being optionally substituted, [symbol]
represents a single or a double bond, and [symbol]
represents the bond between R¹ and the rest of the molecule.

4. The compound of claim 3, wherein R⁸ is H or C₁-C₆ alkyl.

5. The compound of claim 3, wherein one among the couples R³-R⁴, R⁴-R⁵, R⁵-R⁶ and R⁶-R⁷ form together with the carbon atoms to which they are bonded a 6-membered aromatic ring, unsubstituted or substituted with one or more, substituent selected from halogen, OH, NH₂, NH—C₁-C₆alkyl, C₁-C₆ alkyl and O—C₁-C₆ alkyl, the other radicals among R³ to R⁷ being H.

6. The compound of claim 3, wherein R⁹ is one substituent selected in the group consisting of OH and halogen and R¹⁰ is H.

7. The compound of claim 1, wherein R¹ and/or R² is selected from the group consisting of quinolinyl, isoquinolinyl, dihydroquinolinyl, dihydroisoquinolinyl, unsubstituted or substituted with one or more substituent selected from OH, C₁-C₆ alkyl or O—C₁-C₆ alkyl.

8. The compound of claim 1, wherein one of R¹ and R² is an optionally substituted nitrogen-containing heterocyclyl group as defined in claim 1 and the other is H, an optionally substituted C₁-C₆ aliphatic chain or an optionally substituted aryl.

9. The compound of claim 1, wherein L¹ and L² are each independently a methylene group or a linker of formula —CH═.

10. The compound of claim 1, wherein R′ is H.

11. The compound of claim 1, wherein X is an oxygen atom.

12. The compound of claim 1 selected in the group consisting of:

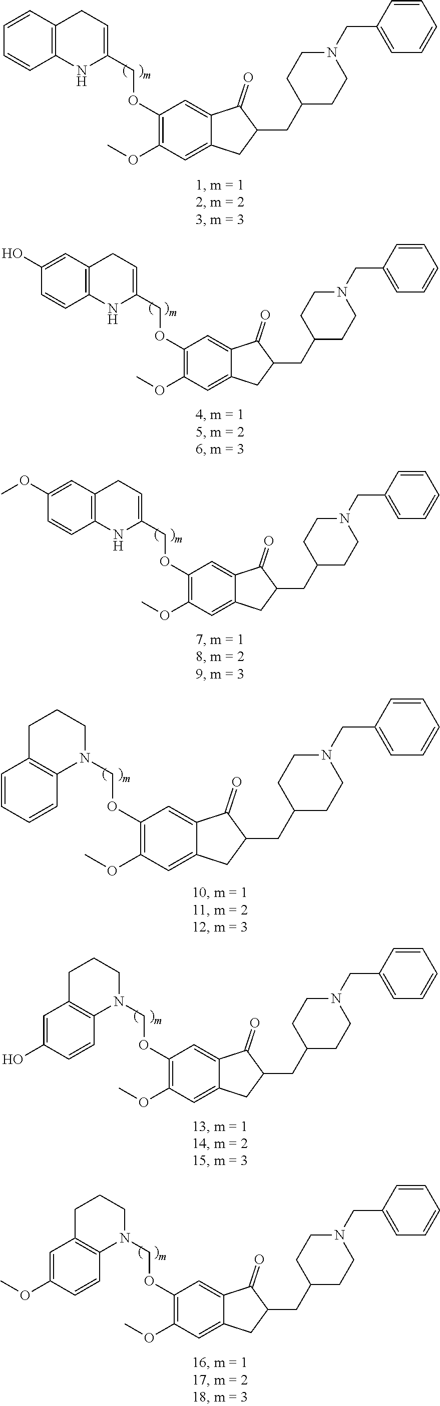

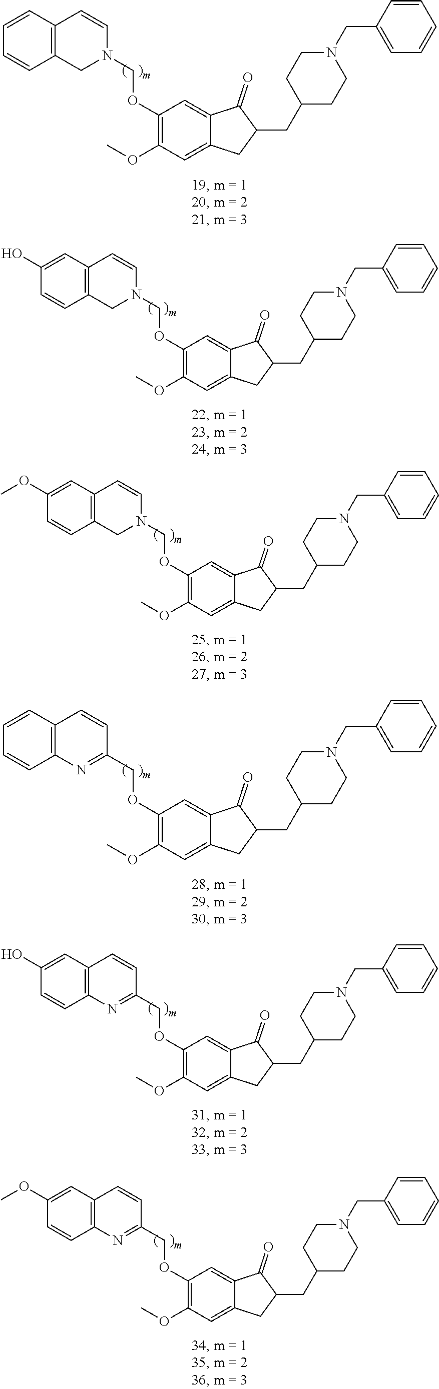

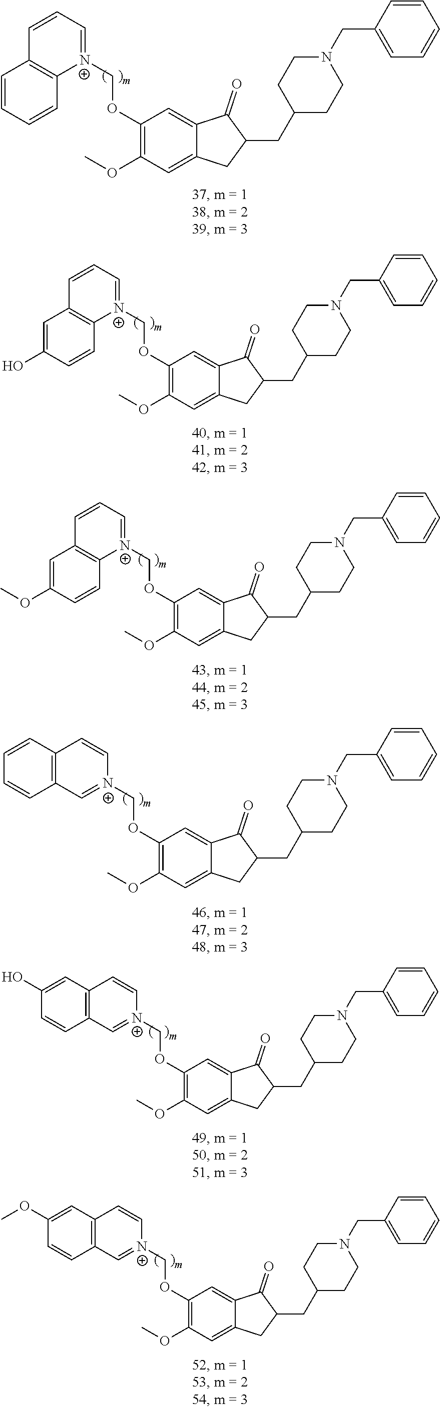

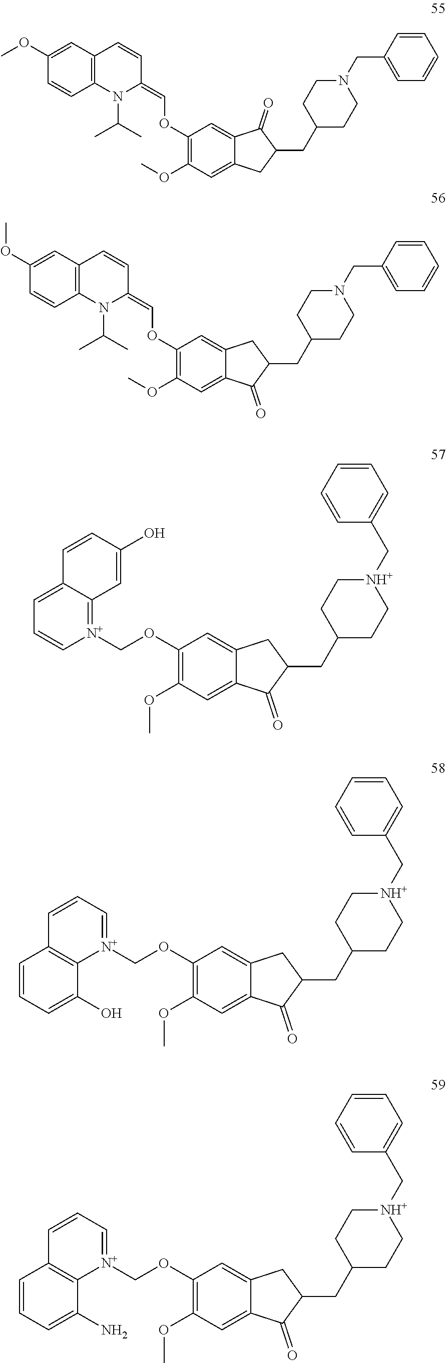 [SMILES: COc1ccc2c(c1)C=C/C(=C/Oc1cc3c(cc1OC)CC(CC1CCN(Cc4ccccc4)CC1)C3=O)N2C(C)C ; COc1ccc2c(c1)C=C/C(=C/Oc1cc3c(cc1OC)C(=O)C(CC1CCN(Cc4ccccc4)CC1)C3)N2C(C)C ; COc1cc2c(cc1OC[n+]1cccc3ccc(O)cc31)CC(CC1CC[NH+](Cc3ccccc3)CC1)C2=O ; COc1cc2c(cc1OC[n+]1cccc3cccc(O)c31)CC(CC1CC[NH+](Cc3ccccc3)CC1)C2=O ; COc1cc2c(cc1OC[n+]1cccc3cccc(N)c31)CC(CC1CC[NH+](Cc3ccccc3)CC1)C2=O]

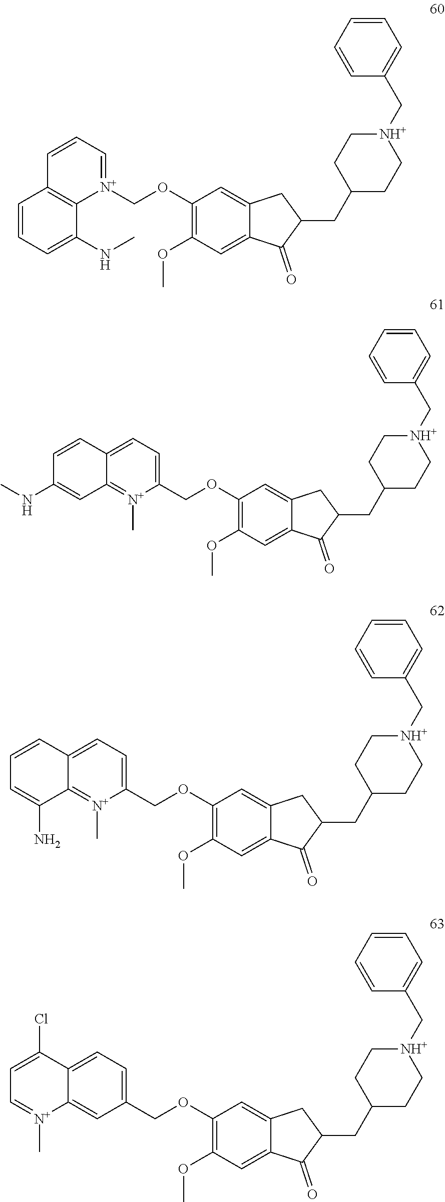 [SMILES: CNc1cccc2ccc[n+](COc3cc4c(cc3OC)C(=O)C(CC3CC[NH+](Cc5ccccc5)CC3)C4)c12 ; CNc1ccc2ccc(COc3cc4c(cc3OC)C(=O)C(CC3CC[NH+](Cc5ccccc5)CC3)C4)[n+](C)c2c1 ; COc1cc2c(cc1OCc1ccc3cccc(N)c3[n+]1C)CC(CC1CC[NH+](Cc3ccccc3)CC1)C2=O ; COc1cc2c(cc1OCc1ccc3c(Cl)cc[n+](C)c3c1)CC(CC1CC[NH+](Cc3ccccc3)CC1)C2=O]

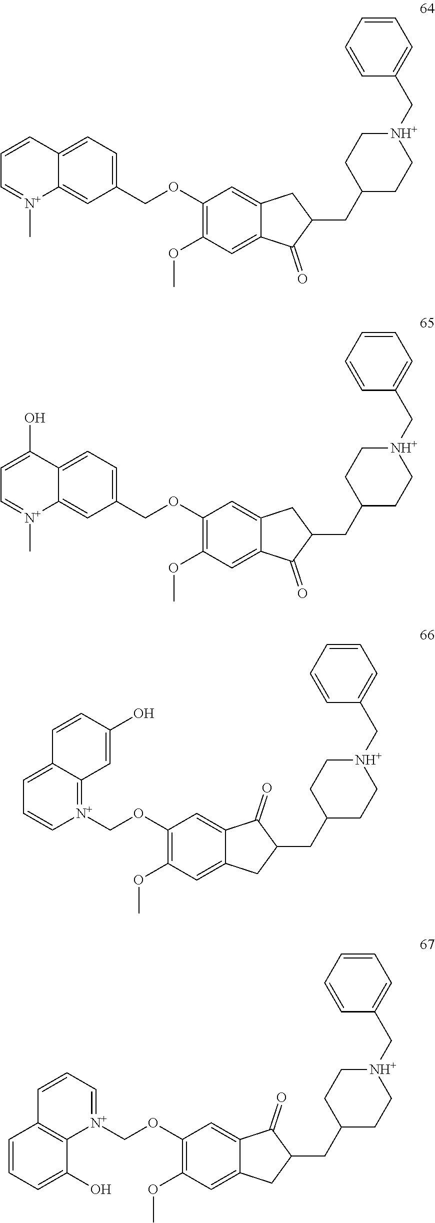 [SMILES: COc1cc2c(cc1OCc1ccc3ccc[n+](C)c3c1)CC(CC1CC[NH+](Cc3ccccc3)CC1)C2=O ; COc1cc2c(cc1OCc1ccc3c(O)cc[n+](C)c3c1)CC(CC1CC[NH+](Cc3ccccc3)CC1)C2=O ; COc1cc2c(cc1OC[n+]1cccc3ccc(O)cc31)C(=O)C(CC1CC[NH+](Cc3ccccc3)CC1)C2 ; COc1cc2c(cc1OC[n+]1cccc3cccc(O)c31)C(=O)C(CC1CC[NH+](Cc3ccccc3)CC1)C2]

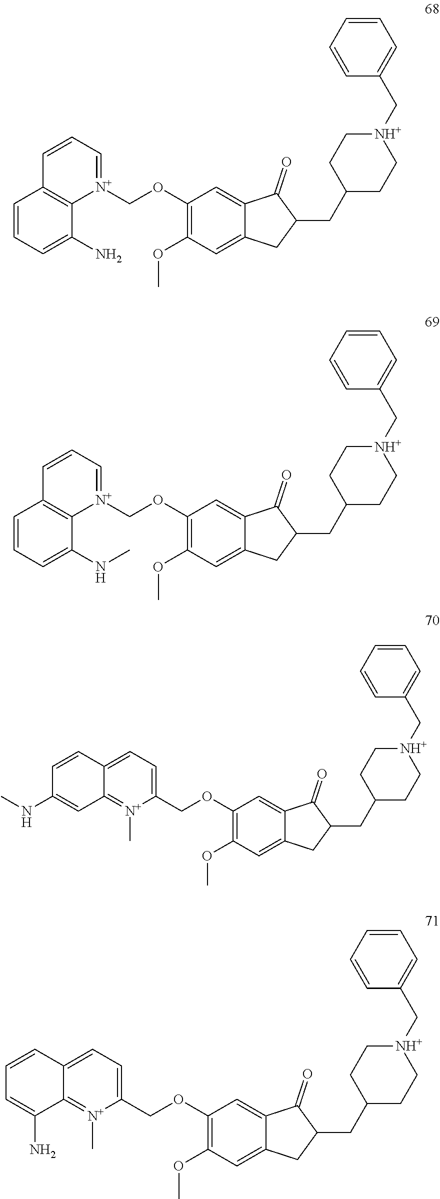 [SMILES: COc1cc2c(cc1OC[n+]1cccc3cccc(N)c31)C(=O)C(CC1CC[NH+](Cc3ccccc3)CC1)C2 ; CNc1cccc2ccc[n+](COc3cc4c(cc3OC)CC(CC3CC[NH+](Cc5ccccc5)CC3)C4=O)c12 ; CNc1ccc2ccc(COc3cc4c(cc3OC)CC(CC3CC[NH+](Cc5ccccc5)CC3)C4=O)[n+](C)c2c1 ; COc1cc2c(cc1OCc1ccc3cccc(N)c3[n+]1C)C(=O)C(CC1CC[NH+](Cc3ccccc3)CC1)C2]

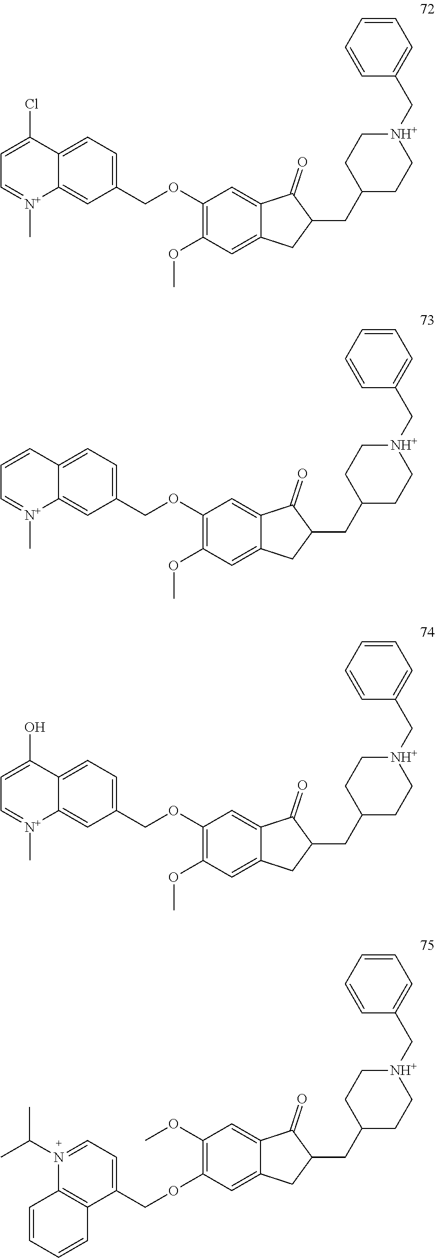

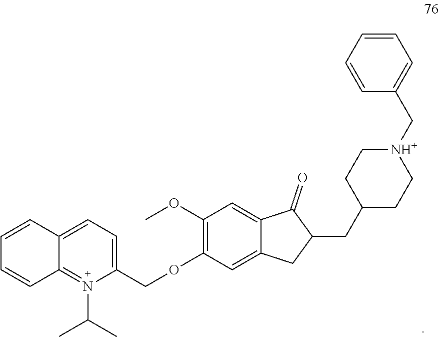

13. A pharmaceutical composition comprising a compound as defined in claim 1 and a pharmaceutically acceptable excipient.

14-15. (canceled)

16. A method for inhibiting acetylcholinesterase comprising the administration to a patient in need thereof of an effective quantity of a compound as defined in claim 1 or a pharmaceutically acceptable salt and/or solvate thereof.

17. A method for preventing and/or treating compulsive disorders and related behaviors, and neurodegenerative disorders comprising the administration to a patient in need thereof of an effective amount of a compound as defined in claim 1 or a pharmaceutically acceptable salt and/or solvate thereof.

18. The method according to claim 17, wherein the compulsive disorders and related behaviors are selected from the group consisting of addiction, obsessive-compulsive disorders and eating disorders; and neurodegenerative disorders are selected from the group consisting of Parkinson's disease and Alzheimer's disease.

19. The method according to claim 18, wherein the eating disorders are selected from the group consisting of anorexia, bulimia and obesity.

20. The compound of claim 4, wherein R⁸ is a methyl.

21. The compound of claim 8, wherein one of R¹ and R² is H, a C₁-C₆ alkyl or a phenyl.

22. The compound of claim 21, wherein one of R¹ and R² is a methyl.
